(19) United States
(12) Patent Application Publication
(10) Pub. No.: US 2006/0166920 A1
(43) Pub. Date: Jul. 27, 2006

(54) Oligonucleotide based therapeutics

(75) Inventors: Liang Xu, Ann Arbor, MI (US); Marc E. Lippman, Ann Arbor, MI (US); Meilan Liu, Ann Arbor, MI (US)

Correspondence Address: Medlen & Carroll, LLP, Suite 350, 101 Howard Street, San Francisco, CA, 94105, US

(73) Assignee: Regents of the University of Michigan, Ann Arbor, MI (US)

(21) Appl. No.: 11/318,799

(22) Filed: Dec. 27, 2005

Related U.S. Application Data
(60) Provisional application No. 60/639,251, filed on Dec. 27, 2004.

(51) Int. Cl. A61K 48/00

(52) U.S. Cl. 514/44

(57) ABSTRACT

The present invention relates to compositions comprising dsRNA. In particular, the present invention provides dsRNA comprising nucleotide sequence that is selectively complementary to bcl-xL mRNA sequence and that is not selectively complementary to bcl-xS mRNA sequence, recombinant nucleic acid comprising a vector and nucleic acid sequence for expressing bcl-xL dsRNA, pharmaceutical compositions comprising bcl-xL dsRNA, kits comprising such compositions, and methods of using the same in research, therapeutic, diagnostic and/or drug screening applications.

This application claims priority to U.S. Provisional Patent Application 60/639,251, filed Dec. 27, 2004, the entire contents of which are hereby incorporated by reference.

This invention was funded, in part, under Department of Defense grants W81XWH-04-1-0215 and DAMD17-03-1-0564. The government may have certain rights in the invention.

### FIELD OF THE INVENTION

The present invention relates to compositions comprising dsRNA. In particular, the present invention provides dsRNA comprising nucleotide sequence that is selectively complementary to bcl-xL mRNA sequence compared to bcl-xS mRNA sequence, recombinant nucleic acid comprising a vector and nucleic acid sequence for expressing bcl-xL dsRNA, pharmaceutical compositions comprising bcl-xL dsRNA, kits comprising such compositions, and methods of using the same in research and/or therapeutic applications.

### BACKGROUND OF THE INVENTION

Many diseases, including cancers, arise from the abnormal, elevated expression or activity of a particular gene (e.g., anti-apoptotic genes such as bcl-xL), a group of genes, or a mutant form of protein. The ability to selectively silence the expression of these genes, while concurrently leaving unaffected the expression of genes that promote cell death holds great therapeutic potential.

A number of therapeutic agents designed to inhibit expression of a target gene have been developed, including antisense ribonucleic acid (RNA) (See, e.g., Skorski, et al., Proc. Natl. Acad. Sci. USA 91:4504 (1994)) and hammerhead-based ribozymes (See, e.g., James, and Gibson, Blood 91:371 (1998)). However, both of these agents have inherent limitations. Antisense approaches, using either single-stranded RNA or DNA, act in a 1:1 stoichiometric relationship and thus have low efficacy (Skorski et al., supra). Hammerhead ribozymes, which because of their catalytic activity can degrade a higher number of target molecules, have been used to overcome the stoichiometry problem associated with antisense RNA. However, hammerhead ribozymes require specific nucleotide sequences in the target gene, sequences that are not always present.

More recently, double-stranded RNA molecules (dsRNA) have been shown to block gene expression in a highly conserved regulatory mechanism known as RNA interference (RNAi). Fire et al. (WO 99/32619) disclose the use of a dsRNA of at least 25 nucleotides in length to inhibit the expression of a target gene in C. elegans. dsRNA has also been shown to degrade target RNA in other organisms, including plants (See, e.g., WO 99/53050 and WO 99/61631), Drosophilia (See, e.g., Yang, et al., Curr. Biol. 10:1191 (2000))), and mammals (See, e.g., WO 00/44895; and DE 101 00 586.5).

In RNA interference, the RNAse III Dicer processes dsRNA into small interfering RNAs (siRNA) of approximately 22 nucleotides, which serve as guide sequences to induce target-specific mRNA cleavage by an RNA-induced silencing complex RISC (Hammond et al., Nature 404:293 (2000)). In other words, RNAi involves a catalytic-type reaction whereby new siRNAs are generated through successive cleavage of long dsRNA. Thus, unlike antisense oligonucleotides, RNAi degrades target RNA in a non-stoichiometric manner. When administered to a cell or organism, exogenous dsRNA has been shown to direct the sequence-specific degradation of endogenous messenger RNA (mRNA) through RNAi.

Despite significant advances in the field, there remains a need for an agent that can selectively and efficiently silence an aberrantly expressed target gene (e.g., an anti-apoptotic gene such as bcl-xL), while concurrently leaving unaffected the expression of genes that promote cell death, using the cell's own RNAi machinery. More specifically, an agent that has high biological activity, in vivo stability, that can effectively inhibit expression of a target anti-apoptotic gene at a low dose and leave unaffected expression of pro-apoptotic genes, would be highly desirable. Compositions comprising such agents would be useful for treating diseases caused by the aberrant expression of anti-apoptotic genes (e.g., bcl-xL). Moreover, the ability to modulate apoptosis in cells using such compositions would be a valuable tool for identification of agents that can modulate apoptosis and/or control cell proliferation and differentiation in research or therapeutic settings.

### SUMMARY OF THE INVENTION

The present invention relates to compositions comprising dsRNA. In particular, the present invention provides dsRNA comprising nucleotide sequence that is selectively complementary to bcl-xL mRNA sequence compared to bcl-xS mRNA sequence, recombinant nucleic acid comprising a vector and nucleic acid sequence for expressing bcl-xL dsRNA, pharmaceutical compositions comprising bcl-xL dsRNA, kits comprising such compositions, and methods of using the same in research and/or therapeutic applications.

Accordingly, the present invention provides a composition comprising dsRNA, wherein the dsRNA comprises a sense RNA strand and an antisense RNA strand, wherein the sense and the antisense RNA strands form an RNA duplex, and wherein the duplex comprises a complementary nucleotide sequence that is selectively complementary to at least a part of bcl-xL mRNA sequence (e.g., SEQ ID NO: 1) or equivalent sequences or equivalent sequences but not selectively complementary to bcl-xS mRNA sequence (e.g., SEQ ID NO: 2) or equivalent sequences. In some embodiments, the complementary nucleotide sequence is substantially identical to a target sequence of about 19 to about 25 contiguous nucleotides in bcl-xL mRNA, and the target sequence is absent in bcl-xS mRNA. In some embodiments, the sense RNA strand comprises one RNA molecule and the antisense RNA strand comprises one RNA molecule. In some embodiments, the RNA duplex comprises at least one linker between the sense and the antisense RNA strands. In some embodiments, the sense and the antisense RNA strands forming the RNA duplex are linked via a single-stranded hairpin. In some embodiments, the linker is a chemical linker. In some embodiments, the dsRNA comprises non-nucleotide material. In some embodiments, the sense and the antisense RNA strands are stabilized against nuclease degradation. In some embodiments, at least one of the RNA strands comprises a nucleotide overhang. In some embodiments, the overhang is a 3′ overhang. In some embodiments, the 3′ overhang comprises from 1 to about 7 nucleotides. In some embodiments, the nucleotide overhang is on the 3′ terminus of the sense RNA strand.

The present invention also provides a recombinant nucleic acid comprising a vector comprising nucleic acid sequence for expressing a dsRNA comprising a sense RNA strand and an antisense RNA strand, wherein the sense and the antisense RNA strands form an RNA duplex, and wherein the duplex comprises a complementary nucleotide sequence that is selectively complementary to at least a part of bcl-xL mRNA sequence (e.g., SEQ ID NO: 1) or equivalent sequences or equivalent sequences but not selectively complementary to bcl-xS mRNA sequence (e.g., SEQ ID NO: 2) or equivalent sequences, and wherein the complementary nucleotide sequence is less than 25 nucleotides in length. In some embodiments, the nucleic acid sequence for expressing the dsRNA is selected from the group consisting of SEQ ID NOS: 3-9. In some embodiments, the vector is selected from the group consisting of a plasmid, an adenoviral vector, an adeno-associated vector, a lentiviral vector, a retroviral vector, and a herpes virus vector. In some embodiments, the nucleic acid sequence for expressing a dsRNA comprises an inducible or regulatable promoter. In some embodiments, the vector comprises a human H1 or human U6 promoter. In some embodiments, the nucleic acid sequence for expressing a dsRNA comprises a sense RNA strand coding sequence in operable combination with i) a human U6 promoter and ii) a poly T termination sequence; and an antisense RNA strand coding sequence in operable combination with i) a human U6 promoter and ii) a poly T termination sequence.

The present invention also provides a pharmaceutical composition comprising a dsRNA and a pharmaceutically acceptable carrier, wherein the dsRNA comprises a sense RNA strand and an antisense RNA strand, wherein the sense and the antisense RNA strands form an RNA duplex, and wherein the duplex comprises a complementary nucleotide sequence that is selectively complementary to at least a part of bcl-xL mRNA sequence (e.g., SEQ ID NO: 1) or equivalent sequences but not selectively complementary to bcl-xS mRNA sequence (e.g., SEQ ID NO: 2) or equivalent sequences. In some embodiments, the pharmaceutical composition further comprises lipofectin, lipofectamine, cellfectin, polycations, liposomes, capsid, capsoid, polymeric nanocapsule and/or a polymeric microcapsule.

The present invention also provides a pharmaceutical composition comprising a recombinant nucleic acid comprising a vector comprising nucleic acid sequence for expressing a dsRNA comprising a sense RNA strand and an antisense RNA strand, wherein the sense and the antisense RNA strands form an RNA duplex, and wherein the duplex comprises a complementary nucleotide sequence that is selectively complementary to at least a part of bcl-xL mRNA sequence (e.g., SEQ ID NO: 1) or equivalent sequences but not selectively complementary to bcl-xS mRNA sequence (e.g., SEQ ID NO: 2) or equivalent sequences, or a physiologically acceptable salt thereof, and a pharmaceutically acceptable carrier. In some embodiments, the carrier is in an aqueous solution. In some embodiments, the aqueous solution is phosphate buffered saline. In some embodiments, the pharmaceutical composition further comprises lipofectin, lipofectamine, cellfectin, polycations, liposomes, capsid, capsoid, polymeric nanocapsule and/or a polymeric microcapsule.

The present invention also provides a method for treating a disease caused by expression of bcl-xL in a mammal, comprising administering to the mammal a dsRNA and a pharmaceutically acceptable carrier, wherein the dsRNA comprises a sense RNA strand and an antisense RNA strand, wherein the sense and the antisense RNA strands form an RNA duplex, and wherein the duplex comprises a complementary nucleotide sequence that is complementary to at least a part of bcl-xL mRNA sequence (e.g., SEQ ID NO: 1) or equivalent sequences but not complementary to bcl-xS mRNA sequence (e.g., SEQ ID NO: 2) or equivalent sequences.

The present invention also provides a method for treating an oncogenic disease in a mammal, comprising administering to the mammal a pharmaceutical composition comprising a recombinant nucleic acid comprising a vector comprising nucleic acid sequence for expressing a dsRNA comprising a sense RNA strand and an antisense RNA strand, wherein the sense and the antisense RNA strands form an RNA duplex, and wherein the duplex comprises a complementary nucleotide sequence that is selectively complementary to at least a part of bcl-xL mRNA sequence (e.g., SEQ ID NO: 1) or equivalent sequences but not selectively complementary to bcl-xS mRNA sequence (e.g., SEQ ID NO: 2) or equivalent sequences, and wherein the complementary nucleotide sequence is less than 25 nucleotides in length, or a physiologically acceptable salt thereof, and a pharmaceutically acceptable carrier. In some embodiments, the disease is selected from the group consisting of acute lymphocytic leukemia, acute myelocytic leukemia, acoustic neuroma, adenocarcinoma, angiosarcoma, astrocytoma, basal cell carcinoma, bile duct carcinoma, bladder carcinoma, bone originated tumor, bone sarcoma, brain tumor, breast cancer, bronchogenic carcinoma, carcinoma, cervical cancer, chondrosarcoma, chordoma, choriocarcinoma, chronic lymphocytic leukemia, colon carcinoma, craniopharyngioma, cystadenocarcinoma, embryonal carcinoma, endotheliosarcoma, ependymoma, epithelial carcinoma, esophageal carcinoma, Ewing's tumor, fibrosarcoma, glioma, heavy chain disease, hemangioblastoma, hepatic carcinoma, hodgkin's lymphoma, leiomyosarcoma, leukemia, liposarcoma, lung carcinoma, lymphangioendotheliosarcoma, lymphangiosarcoma, medullary carcinoma, medulloblastoma, melanoma, meningioma, mesothelioma, multiple myeloma, myxosarcoma, neuroblastoma, non-Hodgkin's lymphoma, pancreatic cancer, oligodendroglioma, osteogenic sarcoma, ovarian cancer, pancreatic carcinoma, papillary carcinoma, papillary adenocarcinoma, pinealoma, polycythemia vera, acute promyelocytic leukemia, prostate cancer, rectal cancer, renal cell carcinoma, retinoblastoma, rhabdomyosarcoma, sarcoma, sebaceous gland carcinoma, seminoma, small cell lung carcinoma, squamous cell carcinoma, stomach carcinoma, synovioma, sweat gland carcinoma, testicular tumor, uterus carcinoma, Waldenstrom's macroglobulinemia, and Wilms' tumor.

The present invention also provides a method of modifying levels of human bcl-xL mRNA, while concurrently unaffecting levels of bcl-xS mRNA, comprising administering to a host or host tissue an effective amount of a dsRNA comprising a sense RNA strand and an antisense RNA strand, wherein the sense and the antisense RNA strands form an RNA duplex, and wherein the duplex comprises a complementary nucleotide sequence that is selectively complementary to at least a part of bcl-xL mRNA sequence (e.g., SEQ ID NO: 1) or equivalent sequences but not selectively complementary to bcl-xS mRNA sequence (e.g., SEQ ID NO: 2) or equivalent sequences. These methods find use in research, diagnostic, drug screening and therapeutic applications. In some embodiments, the modifying reduces levels of bcl-xL mRNA. In some embodiments, the host is a human being. In some embodiments, the effective amount of the dsRNA is from about 1 nM to about 100 nM. In some embodiments, the dsRNA is administered in conjunction with a delivery reagent. In some embodiments, the delivery agent is selected from the group consisting of lipofectin, lipofectamine, cellfectin, polycations, liposomes, capsid, capsoid, polymeric nanocapsule and/or a polymeric microcapsule. In some embodiments, the dsRNA is expressed from a vector. In some embodiments, the vector is selected from the group consisting of a plasmid, an adenoviral vector, an adeno-associated vector, a lentiviral vector, a retroviral vector, and a herpes virus vector. In some embodiments, the vector comprises an inducible or regulatable promoter. In some embodiments, the vector comprises a human H1 or human U6 promoter. In some embodiments, the siRNA is administered by an enteral administration route. In some embodiments, the enteral administration route is selected from the group consisting of oral, rectal, and intranasal. In some embodiments, the dsRNA is administered by a parenteral administration route. In some embodiments, the parenteral administration route is selected from the group consisting of intravascular administration, peri- and intra-tissue administration, subcutaneous injection or deposition, subcutaneous infusion, intraocular administration, and direct application at or near a site of tumorigenesis. In some embodiments, the intravascular administration is selected from the group consisting of intravenous bolus injection, intravenous infusion, intra-arterial bolus injection, intra-arterial infusion and catheter instillation intro the vasculature. In some embodiments, the peri- and intra-tissue injection is selected from the group consisting of peri-tumoral injection, intra-tumoral injection, intra-retinal injection, and subretinal injection. In some embodiments, the direct application at or near the site of tumorigenesis comprises application by catheter, suppository, injection, an implant comprising a porous material, an implant comprising a non-porous material, or an implant comprising a gelatinous material. In some embodiments, the host tissue comprises tumor cells. In some embodiments, the levels of bcl-xL mRNA are reduced by greater than 50%. In some embodiments, the levels of bcl-xL mRNA are reduced by greater than 75%. In some embodiments, the levels of bcl-xL mRNA are reduced by greater than 90%.

The present invention also provides a method for inducing apoptosis in a cell comprising administering to the cell an effective amount of a dsRNA comprising a sense RNA strand and an antisense RNA strand, wherein the sense and the antisense RNA strands form an RNA duplex, and wherein the duplex comprises a complementary nucleotide sequence that is selectively complementary to at least a part of bcl-xL mRNA sequence (e.g., SEQ ID NO: 1) or equivalent sequences but not complementary to bcl-xS mRNA sequence (e.g., SEQ ID NO: 2) or equivalent sequences, and wherein the complementary nucleotide sequence is less than 25 nucleotides in length. In some embodiments, the cell exists in vitro. In some embodiments, the cell exists in vivo in a mammal. In some embodiments, the mammal is a human being.

The present invention also provides a method for inducing apoptosis in a cell comprising administering to the cell a recombinant nucleic acid comprising a vector comprising nucleic acid sequence for expressing a dsRNA comprising a sense RNA strand and an antisense RNA strand, wherein the sense and the antisense RNA strands form an RNA duplex, and wherein the duplex comprises a complementary nucleotide sequence that is selectively complementary to at least a part of bcl-xL mRNA sequence (e.g., SEQ ID NO: 1) or equivalent sequences but not selectively complementary to bcl-xS mRNA sequence (e.g., SEQ ID NO: 2) or equivalent sequences, or a physiologically acceptable salt thereof. In some embodiments, the cell exists in vitro. In some embodiments, the cell exists in vivo in a mammal. In some embodiments, the mammal is a human being.

The present invention also provides a method for inducing apoptosis in a cell comprising administering to the cell a recombinant nucleic acid comprising a vector comprising nucleic acid sequence for expressing a dsRNA comprising a sense RNA strand and an antisense RNA strand, wherein the sense and the antisense RNA strands form an RNA duplex, and wherein the duplex comprises a complementary nucleotide sequence that is selectively complementary to at least a part of bcl-xL mRNA sequence (e.g., SEQ ID NO: 1) or equivalent sequences but not selectively complementary to bcl-xS mRNA sequence (e.g., SEQ ID NO: 2) or equivalent sequences, or a physiologically acceptable salt thereof, and a pharmaceutically acceptable carrier. In some embodiments, the cell exists in vitro. In some embodiments, the cell exists in vivo in a mammal. In some embodiments, the mammal is a human being.

The present invention also provides a method of sensitizing a subject to chemotherapeutic agents comprising administering to the subject a recombinant nucleic acid comprising a vector comprising nucleic acid sequence for expressing a dsRNA comprising a sense RNA strand and an antisense RNA strand, wherein the sense and the antisense RNA strands form an RNA duplex, and wherein the duplex comprises a complementary nucleotide sequence that is selectively complementary to at least a part of bcl-xL mRNA sequence (e.g., SEQ ID NO: 1) or equivalent sequences but not selectively complementary to bcl-xS mRNA sequence (e.g., SEQ ID NO: 2) or equivalent sequences, or a physiologically acceptable salt thereof, and a pharmaceutically acceptable carrier.

The present invention also provides a method of sensitizing a subject to chemotherapeutic agents comprising administering to the subject an effective amount of a pharmaceutical composition comprising a dsRNA comprising a sense RNA strand and an antisense RNA strand, wherein the sense and the antisense RNA strands form an RNA duplex, and wherein the duplex comprises a complementary nucleotide sequence that is selectively complementary to at least a part of bcl-xL mRNA sequence (e.g., SEQ ID NO: 1) or equivalent sequences but not selectively complementary to bcl-xS mRNA sequence (e.g., SEQ ID NO: 2) or equivalent sequences.

The present invention also provides a kit comprising a composition comprising siRNA, wherein the dsRNA comprises a sense RNA strand and an antisense RNA strand, wherein the sense and the antisense RNA strands form an RNA duplex, and wherein the duplex comprises a complementary nucleotide sequence that is selectively complementary to at least a part of bcl-xL mRNA sequence (e.g., SEQ ID NO: 1) or equivalent sequences but not selectively complementary to bcl-xS mRNA sequence (e.g., SEQ ID NO: 2) or equivalent sequences.

The present invention also provides a kit comprising a vector comprising nucleic acid sequence for expressing a dsRNA comprising a sense RNA strand and an antisense RNA strand, wherein the sense and the antisense RNA strands form an RNA duplex, and wherein the duplex comprises a complementary nucleotide sequence that is selectively complementary to at least a part of bcl-xL mRNA sequence (e.g., SEQ ID NO: 1) or equivalent sequences but not selectively complementary to bcl-xS mRNA sequence (e.g., SEQ ID NO: 2) or equivalent sequences.

The present invention also provides a kit comprising a pharmaceutical composition comprising a dsRNA and a pharmaceutically acceptable carrier, wherein the dsRNA comprises a sense RNA strand and an antisense RNA strand, wherein the sense and the antisense RNA strands form an RNA duplex, and wherein the duplex comprises a complementary nucleotide sequence that is selectively complementary to at least a part of bcl-xL mRNA sequence (e.g., SEQ ID NO: 1) or equivalent sequences but not selectively complementary to bcl-xS mRNA sequence (e.g., SEQ ID NO: 2) or equivalent sequences; or a pharmaceutical composition comprising a recombinant nucleic acid comprising a vector comprising nucleic acid sequence for expressing a dsRNA comprising a sense RNA strand and an antisense RNA strand, wherein the sense and the antisense RNA strands form an RNA duplex, and wherein duplex comprises a complementary nucleotide sequence that is selectively complementary to at least a part of bcl-xL mRNA sequence (e.g., SEQ ID NO: 1) or equivalent sequences but not selectively complementary to bcl-xS mRNA sequence (e.g., SEQ ID NO: 2) or equivalent sequences, or a physiologically acceptable salt thereof, and a pharmaceutically acceptable carrier.

The present invention also provides a dsRNA coding sequence that encodes dsRNA that selectively hybridizes to bcl-xL mRNA that does not selectively hybridize to bcl-xS mRNA. In some embodiments, the coding sequence is selected from the group consisting of SEQ ID NOS: 3-9.

The present invention also provides a method of identifying genes involved in apoptosis, comprising administering to cells a composition comprising dsRNA, wherein the dsRNA comprises a sense RNA strand and an antisense RNA strand, wherein the sense and the antisense RNA strands form an RNA duplex, and wherein the duplex comprises a complementary nucleotide sequence that is selectively complementary to at least a part of bcl-xL mRNA sequence (SEQ ID NO: 1) or equivalent sequences but not selectively complementary to bcl-xS mRNA sequence (SEQ ID NO: 2) or equivalent sequences, and monitoring the gene expression profile of the cells. In some embodiments, the cells are stably transfected with a composition comprising nucleic acid encoding the dsRNA.

### DESCRIPTION OF THE DRAWINGS

FIG. 1 depicts Western blot analysis of human prostate cancer PC-3 cells transfected with Bcl-xL siRNA cassettes.

FIG. 2 depicts Western blot analysis of human breast cancer MCF-7 cells transfected with Bcl-xL siRNA cassettes

FIG. 3 depicts MCF-7 cells transfected with Bcl-xL siRNA cassettes 48 hrs after transfection.

FIG. 4 depicts the plasmid psiBcl-xL comprising an siRNA sequence of the present invention.

FIG. 5 depicts the colony formation of MCF-7 cells transfected with psiLuc or psiBcl-xL.

FIG. 6 depicts GFP-fluorescing, MCF-7-psiBcl-xL stable clones.

FIG. 7 depicts Western blot analysis of MCF-7-psiBcl-xL stable clones.

FIG. 8 depicts a cytotoxicity assay of MCF-7 psiBcl-xL stable clones treated with cisplatin (CDDP) and docetaxel (TXT).

### DEFINITIONS

To facilitate an understanding of the present invention, a number of terms and phrases as used herein are defined below:

The terms “protein” and “polypeptide” refer to compounds comprising amino acids joined via peptide bonds and are used interchangeably.

As used herein, “amino acid sequence” is recited herein to refer to an amino acid sequence of a protein molecule. An “amino acid sequence” can be deduced from the nucleic acid sequence encoding the protein. However, terms such as “polypeptide” or “protein” are not meant to limit the amino acid sequence to the deduced amino acid sequence, but include post-translational modifications of the deduced amino acid sequences, such as amino acid deletions, additions, and modifications such as glycolsylations and addition of lipid moieties.

As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity or more (e.g., 99 percent sequence identity). Preferably, residue positions which are not identical differ by conservative amino acid substitutions.

The terms “variant” and “mutant” when used in reference to a polypeptide refer to an amino acid sequence that differs by one or more amino acids from another, usually related polypeptide. The variant may have “conservative” changes, wherein a substituted amino acid has similar structural or chemical properties. One type of conservative amino acid substitutions refers to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine. More rarely, a variant may have “non-conservative” changes (e.g., replacement of a glycine with a tryptophan). Similar minor variations may also include amino acid deletions or insertions (in other words, additions), or both. Guidance in determining which and how many amino acid residues may be substituted, inserted or deleted without abolishing biological activity may be found using computer programs well known in the art, for example, DNAStar software. Variants can be tested in functional assays. Preferred variants have less than 10%, and preferably less than 5%, and still more preferably less than 2% changes (whether substitutions, deletions, and so on).

The term “gene” refers to a nucleic acid (e.g., DNA or RNA) sequence that comprises coding sequences necessary for the production of an RNA, and/or a polypeptide or its precursor (e.g., proinsulin). A functional polypeptide can be encoded by a full length coding sequence or by any portion of the coding sequence as long as the desired activity or functional properties (e.g., enzymatic activity, ligand binding, signal transduction, etc.) of the polypeptide are retained. The term “portion” when used in reference to a gene refers to fragments of that gene. The fragments may range in size from a few nucleotides to the entire gene sequence minus one nucleotide. Thus, “a nucleotide comprising at least a portion of a gene” may comprise fragments of the gene or the entire gene.

The term “gene” may also encompasses the coding regions of a structural gene and includes sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences which are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers which control or influence the transcription of the gene. The 3′ flanking region may contain sequences which direct the termination of transcription, posttranscriptional cleavage and polyadenylation.

The term “heterologous gene” refers to a gene encoding a factor that is not in its natural environment (e.g., has been altered by the hand of man). For example, a heterologous gene includes a gene from one species introduced into another species. A heterologous gene also includes a gene native to an organism that has been altered in some way (e.g., mutated, added in multiple copies, linked to a non-native promoter or enhancer sequence, etc.). Heterologous genes may comprise a gene sequence that comprise cDNA forms of the gene; the cDNA sequences may be expressed in either a sense (to produce mRNA) or anti-sense orientation (to produce an anti-sense RNA transcript that is complementary to the mRNA transcript). Heterologous genes are distinguished from endogenous genes in that the heterologous gene sequences are typically joined to nucleotide sequences comprising regulatory elements such as promoters that are not found naturally associated with the gene for the protein encoded by the heterologous gene or with gene sequences in the chromosome, or are associated with portions of the chromosome not found in nature (e.g., genes expressed in loci where the gene is not normally expressed).

The term “polynucleotide” refers to a molecule comprised of two or more deoxyribonucleotides or ribonucleotides, preferably more than three, and usually more than ten. The exact size will depend on many factors, which in turn depends on the ultimate function or use of the oligonucleotide. The polynucleotide may be generated in any manner, including chemical synthesis, DNA replication, reverse transcription, or a combination thereof. The term “oligonucleotide” generally refers to a short length of single-stranded polynucleotide chain usually less than 30 nucleotides long, although it may also be used interchangeably with the term “polynucleotide.”

The term “nucleic acid” refers to a polymer of nucleotides, or a polynucleotide, as described above. The term is used to designate a single molecule, or a collection of molecules. Nucleic acids may be single stranded or double stranded, and may include coding regions and regions of various control elements, as described below.

The terms “region” or “portion” when used in reference to a nucleic acid molecule refer to a set of linked nucleotides that is less than the entire length of the molecule.

The term “strand” when used in reference to a nucleic acid molecule refers to a set of linked nucleotides which comprises either the entire length or less than or the entire length of the molecule.

The term “links” when used in reference to a nucleic acid molecule refers to a nucleotide region which joins two other regions or portions of the nucleic acid molecule; such connecting means are typically though not necessarily a region of a nucleotide. In a hairpin siRNA molecule, such a linking region may join two other regions of the RNA molecule which are complementary to each other and which therefore can form a double stranded or duplex stretch of the molecule in the regions of complementarity; such links are usually though not necessarily a single stranded nucleotide region contiguous with both strands of the duplex stretch, and are referred to as “loops”.

The term “linker” when used in reference to a multiplex siRNA molecule refers to a connecting means that joins two siRNA molecules. Such connecting means are typically though not necessarily a region of a nucleotide contiguous with a strand of each siRNA molecule; the region of contiguous nucleotide is referred to as a “joining sequence.”

The term “a polynucteotide having a nucleotide sequence encoding a gene” or “a polynucleotide having a nucleotide sequence encoding a gene” or “a nucleic acid sequence encoding” a specified RNA molecule or polypeptide refers to a nucleic acid sequence comprising the coding region of a gene or in other words the nucleic acid sequence which encodes a gene product. The coding region may be present in either a cDNA, genomic DNA or RNA form. When present in a DNA form, the oligonucleotide, polynucleotide, or nucleic acid may be single-stranded (e.g., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors may contain endogenous enhancers/promoters, splice junctions, intervening sequences, polyadenylation signals, etc. or a combination of both endogenous and exogenous control elements.

The term “recombinant” when made in reference to a nucleic acid molecule refers to a nucleic acid molecule that is comprised of segments of nucleic acid joined together by means of molecular biological techniques. The term “recombinant” when made in reference to a protein or a polypeptide refers to a protein molecule that is expressed using a recombinant nucleic acid molecule.

The terms “complementary” and “complementarity” refer to polynucleotides (i.e., a sequence of nucleotides) related by the base-pairing rules. For example, for the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in detection methods that depend upon binding between nucleic acids. This is also of importance in efficacy of siRNA inhibition of gene expression or of RNA function.

As used herein, the terms “selectively complementary” and “selectively hybridizes” are structurally and functionally defined terms that refer to nucleotide sequences that associate with one or more nucleic acid sequences but that function to degrade, in the context of siRNA complexes, only the “selective” mRNA sequences. For example, a complementary nucleotide sequence that is “selectively complementary” to at least a part of bcl-xL mRNA sequence but not “selectively complementary” to bcl-xS mRNA sequence is a sequence that, although it may associate with sequences found in both bcl-xL and bcl-xS mRNA, functions, in the siRNA context of the present invention, to degrade only bcl-xL mRNA sequence, while having no effect on levels of bcl-xS mRNA.

The term “homology” when used in relation to nucleic acids refers to a degree of complementarity. There may be partial homology or complete homology (i.e., identity). As used herein and as known in the art, the term “identity” is the relationship between two or more polynucleotide sequences, as determined by comparing the sequences. Identity also means the degree of sequence relatedness between polynucleotide sequences, as determined by the match between strings of such sequences. Identity can be readily calculated (see, e.g., Computation Molecular Biology, Lesk, A. M., eds., Oxford University Press, New York (1998), and Biocomputing: Informatics and Genome Projects, Smith, D. W., ed., Academic Press, New York (1993), both of which are incorporated by reference herein). While there exist a number of methods to measure identity between two polynucleotide sequences, the term is well known to skilled artisans (see, e.g., Sequence Analysis in Molecular Biology, von Heinje, G., Academic Press (1987); and Sequence Analysis Primer, Gribskov., M. and Devereux, J., eds., M. Stockton Press, New York (1991)). Methods commonly employed to determine identity between sequences include, for example, those disclosed in Carillo, H., and Lipman, D., SIAM J. Applied Math. (1988) 48:1073. “Substantially identical,” as used herein, means there is a very high degree of homology (preferably 100% sequence identity) between the sense strand of the dsRNA and the corresponding part of the target gene. However, dsRNA having greater than 90%, or 95% sequence identity may be used in the present invention, and thus sequence variations that might be expected due to genetic mutation, strain polymorphism, or evolutionary divergence can be tolerated. Although 100% identity is preferred, the dsRNA may contain single or multiple base-pair random mismatches between the RNA and the target gene. As used herein, “target sequence” refers to a sequence of about 19 to about 25 contiguous nucleotides present in bcl-xL mRNA, that, because of alternative splicing of the gene, is absent in bcl-xS mRNA sequence.

The term “complementary RNA strand” (also referred to herein as the “antisense strand”) refers to the strand of a dsRNA present in an RNA duplex that is complementary to an mRNA transcript that is formed during expression of the target gene, or its processing products. As used herein, the term “complementary nucleotide sequence” refers to the region on the complementary RNA strand that is complementary to an mRNA transcript of a portion bcl-xL mRNA. The sequence may include sequence that is found in both bcl-xL and bcl-xS sequence. However, as stated above, the complementary nucleotide sequence may be “selectively complementary,” that is, the nucleotide sequence may be complementary to and therefore associate with one or more nucleic acid sequences, but it will function to degrade, in the context of siRNA complexes, only the “selective” mRNA sequences.

As used herein, the term “dsRNA” refers to a ribonucleic acid molecule having a duplex structure comprising two complementary and anti-parallel nucleic acid strands. Not all nucleotides of a dsRNA must exhibit Watson-Crick base pairs; the two RNA strands may be substantially complementary (i.e., having no more than one or two nucleotide mismatches). The maximum number of base pairs is the number of nucleotides in the shortest strand of the dsRNA. The RNA strands may have the same or a different number of nucleotides. The dsRNA is less than 70, preferably less than 25, and most preferably between 21 and 24 nucleotides in length. dsRNAs of this length are particularly efficient in inhibiting the expression of the target anti-apoptotic gene. “Introducing into” means uptake or absorption in the cell, as is understood by those skilled in the art. Absorption or uptake of dsRNA can occur through cellular processes, or by auxiliary agents or devices. For example, for in vivo delivery, dsRNA can be injected into a tissue site or administered systemically. In vitro delivery includes methods known in the art such as electroporation and lipofection.

As used herein, a “nucleotide overhang” refers to the unpaired nucleotide or nucleotides that protrude from the duplex structure when a 3′-end of one RNA strand extends beyond the 5′-end of the other strand, or vice versa.

As used herein, the terms “cancer,” “hyperproliferative,” “tumor cells,” and “neoplastic” refer to cells having the capacity for autonomous growth, i.e., an abnormal state of condition characterized by rapidly proliferating cell growth. These terms are meant to include all types of cancerous growths or oncogenic processes, metastatic tissues or malignantly transformed cells, tissues, or organs, irrespective of histopathologic type or stage of invasiveness. Proliferative disorders also include hematopoietic neoplastic disorders, including diseases involving hyperplastic/neoplatic cells of hematopoietic origin, e.g., arising from myeloid, lymphoid or erythroid lineages, or precursor cells thereof. Examples of cellular proliferative and/or differentiative disorders include cancer, e.g., carcinoma, sarcoma, metastatic disorders or hematopoietic neoplastic disorders, e.g., leukemias. A metastatic tumor can arise from a multitude of primary tumor types, including but not limited to those of pancreas, prostate, colon, lung, breast and liver origin.

As used herein, the term “treatment” refers to the application or administration of a therapeutic agent to a patient, or application or administration of a therapeutic agent to an isolated tissue or cell line from a patient, who has a disorder, e.g., a disease or condition, a symptom of disease, or a predisposition toward a disease, with the purpose to cure, heal, alleviate, relieve, alter, remedy, ameliorate, improve, or affect the disease, the symptoms of disease, or the predisposition toward disease.

As used herein, a “pharmaceutical composition” comprises a pharmacologically effective amount of a dsRNA and a pharmaceutically acceptable carrier. As used herein, “pharmacologically effective amount,” “therapeutically effective amount” or simply “effective amount” refers to that amount of an RNA effective to produce the intended pharmacological, therapeutic or preventive result. For example, if a given clinical treatment is considered effective when there is at least a 25% reduction in a measurable parameter associated with a disease or disorder, a therapeutically effective amount of a drug for the treatment of that disease or disorder is the amount necessary to effect at least a 25% reduction in that parameter.

The term “pharmaceutically acceptable carrier” refers to a carrier for administration of a therapeutic agent. Such carriers include, but are not limited to, saline, buffered saline, dextrose, water, glycerol, ethanol, and combinations thereof. The term specifically excludes cell culture medium. For drugs administered orally, pharmaceutically acceptable carriers include, but are not limited to pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract.

As used herein, a “transformed cell” is a cell into which a dsRNA molecule has been introduced by means of recombinant DNA techniques.

The following terms are used to describe the sequence relationships between two or more polynucleotides: “reference sequence”, “sequence identity”, “percentage of sequence identity”, and “substantial identity”. A “reference sequence” is a defined sequence used as a basis for a sequence comparison; a reference sequence may be a subset of a larger sequence, for example, as a segment of a full-length cDNA sequence given in a sequence listing or may comprise a complete gene sequence. Generally, a reference sequence is at least 20 nucleotides in length, frequently at least 25 nucleotides in length, and often at least 50 nucleotides in length. Since two polynucleotides may each (1) comprise a sequence (i.e., a portion of the complete polynucleotide sequence) that is similar between the two polynucleotides, and (2) may further comprise a sequence that is divergent between the two polynucleotides, sequence comparisons between-two (or more) polynucleotides are typically performed by comparing sequences of the two polynucleotides over a “comparison window” to identify and compare local regions of sequence similarity. A “comparison window”, as used herein, refers to a conceptual segment of at least 20 contiguous nucleotide positions wherein a polynucleotide sequence may be compared to a reference sequence of at least 20 contiguous nucleotides and wherein the portion of the polynucleotide sequence in the comparison window may comprise additions or deletions (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman (Smith and Waterman, Adv. Appl. Math. 2: 482 (1981)) by the homology alignment algorithm of Needleman and Wunsch (Needleman and Wunsch, J. Mol. Biol. 48:443 (1970)), by the search for similarity method of Pearson and Lipman (Pearson and Lipman, Proc. Natl. Acad. Sci. (U.S.A.) 85:2444 (1988)), by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of homology over the comparison window) generated by the various methods is selected. The term “sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. The terms “substantial identity” as used herein denotes a characteristic of a polynucleotide sequence, wherein the polynucleotide comprises a sequence that has at least 85 percent sequence identity, preferably at least 90 to 95 percent sequence identity, more usually at least 99 percent sequence identity as compared to a reference sequence over a comparison window of at least 20 nucleotide positions, frequently over a window of at least 25-50 nucleotides, wherein the percentage of sequence identity is calculated by comparing the reference sequence to the polynucleotide sequence which may include deletions or additions which total 20 percent or less of the reference sequence over the window of comparison. The reference sequence may be a subset of a larger sequence, for example, as a segment of the full-length sequences of the compositions claimed in the present invention.

When used in reference to a double-stranded nucleic acid sequence such as a cDNA or genomic clone, the term “substantially homologous” refers to any probe that can hybridize to either or both strands of the double-stranded nucleic acid sequence under conditions of low to high stringency as described above.

When used in reference to a single-stranded nucleic acid sequence, the term “substantially homologous” refers to any probe that can hybridize (i.e., it is the complement of) the single-stranded nucleic acid sequence under conditions of low to high stringency as described above.

The term “hybridization” refers to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

The term “Tₘ” refers to the “melting temperature” of a nucleic acid. The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41(% G+C), when a nucleic acid is in aqueous solution at 1 M NaCl (See e.g., Anderson and Young, Quantitative Filter Hybridization, in Nucleic Acid Hybridization (1985)). Other references include more sophisticated computations that take structural as well as sequence characteristics into account for the calculation of Tₘ.

As used herein the term “stringency” refers to the conditions of temperature, ionic strength, and the presence of other compounds such as organic solvents, under which nucleic acid hybridizations are conducted. With “high stringency” conditions, nucleic acid base pairing will occur only between nucleic acid fragments that have a high frequency of complementary base sequences. Thus, conditions of “low” stringency are often required with nucleic acids that are derived from organisms that are genetically diverse, as the frequency of complementary sequences is usually less.

“Low stringency conditions” when used in reference to nucleic acid hybridization comprise conditions equivalent to binding or hybridization at 42 degree C. in a solution consisting of 5 times SSPE (43.8 g/l NaCl, 6.9 g/l NaH.sub.2PO.sub.4.H.sub.2O and 1.85 g/l EDTA, pH adjusted to 7.4 with NaOH), 0.1% SDS, 5 times Denhardt's reagent (50 times Denhardt's contains per 500 ml: 5 g Ficoll (Type 400, Pharmacia), 5 g BSA (Fraction V; Sigma)) and 100 μg/ml denatured salmon sperm DNA followed by washing in a solution comprising 5 times SSPE, 0.1% SDS at 42 degree C. when a probe of about 500 nucleotides in length is employed.

“Medium stringency conditions” when used in reference to nucleic acid hybridization comprise conditions equivalent to binding or hybridization at 42 degree C. in a solution consisting of 5 times SSPE (43.8 g/l NaCl, 6.9 g/l NaH.sub.2PO.sub.4.H.sub.2O and 1.85 g/l EDTA, pH adjusted to 7.4 with NaOH), 0.5% SDS, 5 times Denhardt's reagent and 100 μg/ml denatured salmon sperm DNA followed by washing in a solution comprising 1.0 times SSPE, 1.0% SDS at 42 degree C. when a probe of about 500 nucleotides in length is employed.

“High stringency conditions” when used in reference to nucleic acid hybridization comprise conditions equivalent to binding or hybridization at 42 degree C. in a solution consisting of 5 times SSPE (43.8 g/l NaCl, 6.9 g/l NaH2PO.sub.4.H.sub.20 and 1.85 g/l EDTA, pH adjusted to 7.4 with NaOH), 0.5% SDS, 5 times Denhardt's reagent and 100. μg/ml denatured salmon sperm DNA followed by washing in a solution comprising 0.1 times SSPE, 1.0% SDS at 42 degree C. when a probe of about 500 nucleotides in length is employed.

It is well known that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.).

The term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and, where the RNA encodes a protein, into protein, through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

The term “RNA function” refers to the role of an RNA molecule in a cell. For example, the function of mRNA is translation into a protein. Other RNAs are not translated into a protein, and have other functions; such RNAs include but are not limited to transfer RNA (tRNA), ribosomal RNA (rRNA), and small nuclear RNAs (snRNAs). An RNA molecule may have more than one role in a cell.

The term “inhibition” when used in reference to gene expression or RNA function refers to a decrease in the level of gene expression or RNA function as the result of some interference with or interaction with gene expression or RNA function as compared to the level of expression or function in the absence of the interference or interaction. The inhibition may be complete, in which there is no detectable expression or function, or it may be partial. Partial inhibition can range from near complete inhibition to near absence of inhibition; typically, inhibition is at least about 50% inhibition, or at least about 80% inhibition, or at least about 90% inhibition.

The terms “in operable combination”, “in operable order” and “operably linked” refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

The term “regulatory element” refers to a genetic element that controls some aspect of the expression of nucleic acid sequences. For example, a promoter is a regulatory element that facilitates the initiation of transcription of an operably linked coding region. Other regulatory elements are splicing signals, polyadenylation signals, termination signals, etc.

Transcriptional control signals in eukaryotes comprise “promoter” and “enhancer” elements. Promoters and enhancers consist of short arrays of DNA sequences that interact specifically with cellular proteins involved in transcription (Maniatis, et al., Science 236:1237, 1987). Promoter and enhancer elements have been isolated from a variety of eukaryotic sources including genes in yeast, insect, mammalian and plant cells. Promoter and enhancer elements have also been isolated from viruses and analogous control elements, such as promoters, are also found in prokaryotes. The selection of a particular promoter and enhancer depends on the cell type used to express the protein of interest. Some eukaryotic promoters and enhancers have a broad host range while others are functional in a limited subset of cell types (for review, see Voss, et al., Trends Biochem. Sci., 11:287, 1986; and Maniatis, et al., supra 1987).

The terms “promoter element,” “promoter,” or “promoter sequence” as used herein, refer to a DNA sequence that is located at the 5′ end (i.e. precedes) the protein coding region of a DNA polymer. The location of most promoters known in nature precedes the transcribed region. The promoter functions as a switch, activating the expression of a gene. If the gene is activated, it is said to be transcribed, or participating in transcription. Transcription involves the synthesis of mRNA from the gene. The promoter, therefore, serves as a transcriptional regulatory element and also provides a site for initiation of transcription of the gene into mRNA.

Promoters may be tissue specific or cell specific. The term “tissue specific” as it applies to a promoter refers to a promoter that is capable of directing selective expression of a nucleotide sequence of interest to a specific type of tissue in the relative absence of expression of the same nucleotide sequence of interest in a different type of tissue. Tissue specificity of a promoter may be evaluated by, for example, operably linking a reporter gene to the promoter sequence to generate a reporter construct, introducing the reporter construct into the genome of a plant such that the reporter construct is integrated into every tissue of the resulting transgenic plant, and detecting the expression of the reporter gene (e.g., detecting mRNA, protein, or the activity of a protein encoded by the reporter gene) in different tissues of the transgenic plant. The detection of a greater level of expression of the reporter gene in one or more tissues relative to the level of expression of the reporter gene in other tissues shows that the promoter is specific for the tissues in which greater levels of expression are detected. The term “cell type specific” as applied to a promoter refers to a promoter that is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., immunohistochemical staining. Briefly, tissue sections are embedded in paraffin, and paraffin sections are reacted with a primary antibody that is specific for the polypeptide product encoded by the nucleotide sequence of interest whose expression is controlled by the promoter. A labeled (e.g., peroxidase conjugated) secondary antibody that is specific for the primary antibody is allowed to bind to the sectioned tissue and specific binding detected (e.g., with avidin/biotin) by microscopy.

Promoters may be constitutive or regulatable. The term “constitutive” when made in reference to a promoter means that the promoter is capable of directing transcription of an operably linked nucleic acid sequence in the absence of a stimulus (e.g., heat shock, chemicals, light, etc.). Typically, constitutive promoters are capable of directing expression of a transgene in substantially any cell and any tissue.

In contrast, a “regulatable” or “inducible” promoter is one which is capable of directing a level of transcription of an operably linked nuclei acid sequence in the presence of a stimulus (e.g., heat shock, chemicals, light, etc.) which is different from the level of transcription of the operably linked nucleic acid sequence in the absence of the stimulus.

The enhancer and/or promoter may be “endogenous” or “exogenous” or “heterologous.” An “endogenous” enhancer or promoter is one that is naturally linked with a given gene in the genome. An “exogenous” or “heterologous” enhancer or promoter is one that is placed in juxtaposition to a gene by means of genetic manipulation (i.e., molecular biological techniques) such that transcription of the gene is directed by the linked enhancer or promoter. For example, an endogenous promoter in operable combination with a first gene can be isolated, removed, and placed in operable combination with a second gene, thereby making it a “heterologous promoter” in operable combination with the second gene. A variety of such combinations are contemplated (e.g., the first and second genes can be from the same species, or from different species.

The presence of “splicing signals” on an expression vector often results in higher levels of expression of the recombinant transcript in eukaryotic host cells. Splicing signals mediate the removal of introns from the primary RNA transcript and consist of a splice donor and acceptor site (Sambrook, et al., Molecular Cloning: A Laboratory Manual, 2nd ed., Cold Spring Harbor Laboratory Press, New York (1989) pp. 16.7-16.8). A commonly used splice donor and acceptor site is the splice junction from the 16S RNA of SV40.

Efficient expression of recombinant DNA sequences in eukaryotic cells often requires expression of signals directing the efficient termination and polyadenylation of the resulting transcript. Transcription termination signals are generally found downstream of the polyadenylation signal and are a few hundred nucleotides in length. The term “poly(A) site” or “poly(A) sequence” as used herein denotes a DNA sequence which directs both the termination and polyadenylation of the nascent RNA transcript. Efficient polyadenylation of the recombinant transcript is desirable, as transcripts lacking a poly(A) tail are unstable and are rapidly degraded. The poly(A) signal utilized in an expression vector may be “heterologous” or “endogenous.” An endogenous poly(A) signal is one that is found naturally at the 3′ end of the coding region of a given gene in the genome. A heterologous poly(A) signal is one which has been isolated from one gene and positioned 3′ to another gene. A commonly used heterologous poly(A) signal is the SV40 poly(A) signal. The SV40 poly(A) signal is contained on a 237 bp BamHI/BclI restriction fragment and directs both termination and polyadenylation (Sambrook, supra, at 16.6-16.7).

The term “vector” refers to nucleic acid molecules that transfer DNA segment(s) from one cell to another. The term “vehicle” is sometimes used interchangeably with “vector.” A vector may be used to transfer an expression cassette into a cell; in addition or alternatively, a vector may comprise additional genes, including but not limited to genes which encode marker proteins, by which cell transfection can be determined, selection proteins, be means of which transfected cells may be selected from nontransfected cells, or reporter proteins, by means of which an effect on expression or activity or function of the reporter protein can be monitored.

The term “expression cassette” refers to a chemically synthesized or recombinant DNA molecule containing a desired coding sequence and appropriate nucleic acid sequences necessary for the expression of the operably linked coding sequence either in vitro or in vivo. Expression in vitro includes expression in transcription systems and in transcription/translation systems. Expression in vivo includes expression in a particular host cell and/or organism. Nucleic acid sequences necessary for expression in prokaryotic cell or in vitro expression system usually include a promoter, an operator (optional), and a ribosome binding site, often along with other sequences. Eukaryotic in vitro transcription systems and cells are known to utilize promoters, enhancers, and termination and polyadenylation signals. Nucleic acid sequences necessary for expression via bacterial RNA polymerases, referred to as a transcription template in the art, include a template DNA strand which has a polymerase promoter region followed by the complement of the RNA sequence desired. In order to create a transcription template, a complementary strand is annealed to the promoter portion of the template strand.

The term “expression vector” refers to a vector comprising one or more expression cassettes. Such expression cassettes include those of the present invention, where expression results in an siRNA transcript.

The term “transfection” refers to the introduction of foreign DNA into cells. Transfection may be accomplished by a variety of means known to the art including calcium phosphate-DNA co-precipitation, DEAE-dextran-mediated transfection, polybrene-mediated transfection, glass beads, electroporation, microinjection, liposome fusion, lipofection, protoplast fusion, bacterial infection, viral infection, biolistics (i.e., particle bombardment) and the like. The terms “transfect” and “transform” (and grammatical equivalents, such as “transfected” and “transformed”) are used interchangeably.

The term “stable transfection” or “stably transfected” refers to the introduction and integration of foreign DNA into the genome of the transfected cell. The term “stable transfectant” refers to a cell that has stably integrated foreign DNA into the genomic DNA.

The term “transient transfection” or “transiently transfected” refers to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes. The term “transient transfectant” refers to cells that have taken up foreign DNA but have failed to integrate this DNA.

The terms “bombarding, “bombardment,” and “biolistic bombardment” refer to the process of accelerating particles towards a target biological sample (e.g., cell, tissue, etc.) to effect wounding of the cell membrane of a cell in the target biological sample and/or entry of the particles into the target biological sample. Methods for biolistic bombardment are known in the art (e.g., U.S. Pat. No. 5,584,807, the contents of which are incorporated herein by reference), and are commercially available (e.g., the helium gas-driven microprojectile accelerator (PDS-1000/He, BioRad).

The term “transgene” as used herein refers to a foreign gene that is placed into an organism by introducing the foreign gene into newly fertilized eggs or early embryos. The term “foreign gene” refers to any nucleic acid (e.g., gene sequence) that is introduced into the genome of an animal by experimental manipulations and may include gene sequences found in that animal so long as the introduced gene does not reside in the same location as does the naturally-occurring gene.

The term “host cell” refers to any cell capable of replicating and/or transcribing and/or translating a heterologous gene. Thus, a “host cell” refers to any eukaryotic or prokaryotic cell (e.g., bacterial cells such as E. Coli, yeast cells, mammalian cells, avian cells, amphibian cells, plant cells, fish cells, and insect cells), whether located in vitro or in vivo. For example, host cells may be located in a transgenic animal.

The terms “transformants” or “transformed cells” include the primary transformed cell and cultures derived from that cell without regard to the number of transfers. All progeny may not be precisely identical in DNA content, due to deliberate or inadvertent mutations. Mutant progeny that have the same functionality as screened for in the originally transformed cell are included in the definition of transformants.

The term “selectable marker” refers to a gene which encodes an enzyme having an activity that confers resistance to an antibiotic or drug upon the cell in which the selectable marker is expressed, or which confers expression of a trait which can be detected (e.g., luminescence or fluorescence). Selectable markers may be “positive” or “negative.” Examples of positive selectable markers include the neomycin phosphotrasferase (NPTII) gene that confers resistance to G418 and to kanamycin, and the bacterial hygromycin phosphotransferase gene (hyg), which confers resistance to the antibiotic hygromycin. Negative selectable markers encode an enzymatic activity whose expression is cytotoxic to the cell when grown in an appropriate selective medium. For example, the HSV-tk gene is commonly used as a negative selectable marker. Expression of the HSV-tk gene in cells grown in the presence of gancyclovir or acyclovir is cytotoxic; thus, growth of cells in selective medium containing gancyclovir or acyclovir selects against cells capable of expressing a functional HSV TK enzyme.

The term “reporter gene” refers to a gene encoding a protein that may be assayed. Examples of reporter genes include, but are not limited to, luciferase (See, e.g., deWet et al., Mol. Cell. Biol. 7:725 (1987) and U.S. Pat Nos. 6,074,859; 5,976,796; 5,674,713; and 5,618,682; all of which are incorporated herein by reference), green fluorescent protein (e.g., GenBank Accession Number U43284; a number of GFP variants are commercially available from ClonTech Laboratories, Palo Alto, Calif.), chloramphenicol acetyltransferase, .beta.-galactosidase, alkaline phosphatase, and horse radish peroxidase.

The term “wild-type” when made in reference to a gene refers to a gene that has the characteristics of a gene isolated from a naturally occurring source. The term “wild-type” when made in reference to a gene product refers to a gene product that has the characteristics of a gene product isolated from a naturally occurring source. The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring. A wild-type gene is that which is most frequently observed in a population and is thus arbitrarily designated the “normal” or “wild-type” form of the gene. In contrast, the term “modified” or “mutant” when made in reference to a gene or to a gene product refers, respectively, to a gene or to a gene product which displays modifications in sequence and/or functional properties (i.e., altered characteristics) when compared to the wild-type gene or gene product. It is noted that naturally-occurring mutants can be isolated; these are identified by the fact that they have altered characteristics when compared to the wild-type gene or gene product.

The term “antisense” when used in reference to DNA refers to a sequence that is complementary to a sense strand of a DNA duplex. A “sense strand” of a DNA duplex refers to a strand in a DNA duplex that is transcribed by a cell in its natural state into a “sense mRNA.” Thus an “antisense” sequence is a sequence having the same sequence as the non-coding strand in a DNA duplex. The term “antisense RNA” refers to a RNA transcript that is complementary to all or part of a target primary transcript or mRNA and that blocks the expression of a target gene by interfering with the processing, transport and/or translation of its primary transcript or mRNA. The complementarity of an antisense RNA may be with any part of the specific gene transcript (e.g., at the 5′ non-coding sequence, 3′ non-coding sequence, introns, or the coding sequence). In addition, as used herein, antisense RNA may contain regions of ribozyme sequences that increase the efficacy of antisense RNA to block gene expression. “Ribozyme” refers to a catalytic RNA and includes sequence-specific endoribonucleases.

The term “siRNAs” refers to short interfering RNAs. In some embodiments, siRNAs comprise a duplex, or double-stranded region, of about 18-25 nucleotides long; often siRNAs contain from about two to four unpaired nucleotides at the 3′ end of each strand. At least one strand of the duplex or double-stranded region of a siRNA is substantially homologous to or substantially complementary to a target RNA molecule. siRNAs may also contain additional sequences; non-limiting examples of such sequences include linking sequences, or loops, as well as stem and other folded structures. siRNAs appear to function as key intermediaries in triggering RNA interference in invertebrates and in vertebrates, and in triggering sequence-specific RNA degradation during posttranscriptional gene silencing in plants.

The term “target RNA molecule” refers to an RNA molecule to which at least one strand of the short double-stranded region of an siRNA is homologous or complementary. Typically, when such homology or complementary is about 100%, the siRNA is able to silence or inhibit expression of the target RNA molecule. Although it is believed that processed mRNA is a target of siRNA, the present invention is not limited to any particular hypothesis, and such hypotheses are not necessary to practice the present invention. Thus, it is contemplated that other RNA molecules may also be targets of siRNA. Such targets include unprocessed mRNA, ribosomal RNA, and viral RNA genomes.

The term “ds siRNA” refers to a siRNA molecule that comprises two separate unlinked strands of RNA which form a duplex structure, such that the siRNA molecule comprises two RNA polynucleotides.

The term “hairpin siRNA” refers to a siRNA molecule that comprises at least one duplex region where the strands of the duplex are connected or contiguous at one or both ends, such that the siRNA molecule comprises a single RNA polynucleotide. The antisense sequence, or sequence which is complementary to a target RNA, is a part of the at least one double stranded region.

The term “full hairpin siRNA” refers to a hairpin siRNA that comprises a duplex or double stranded region of about 18-25 base pairs long, where the two strands are joined at one end by a linking sequence, or loop. At least one strand of the duplex region is an antisense strand, and either strand of the duplex region may be the antisense strand. The region linking the strands of the duplex, also referred to as a loop, comprises at least three nucleotides. The sequence of the loop may also a part of the antisense strand of the duplex region, and thus is itself complementary to a target RNA molecule.

The term “partial hairpin siRNA” refers to a hairpin siRNA which comprises an antisense sequence (or a region or strand complementary to a target RNA) of about 18-25 bases long, and which forms less than a full hairpin structure with the antisense sequence. In some embodiments, the antisense sequence itself forms a duplex structure of some or most of the antisense sequence. In other embodiments, the siRNA comprises at least one additional contiguous sequence or region, where at least part of the additional sequence(s) is complementary to part of the antisense sequence.

The term “mismatch” when used in reference to siRNAs refers to the presence of a base in one strand of a duplex region of which at least one strand of an siRNA is a member, where the mismatched base does not pair with the corresponding base in the complementary strand, where pairing is determined by the general base-pairing rules. The term “mismatch” also refers to the presence of at least one additional base in one strand of a duplex region of which at least one strand of an siRNA is a member, where the mismatched base does not pair with any base in the complementary strand, or to a deletion of at least one base in one strand of a duplex region which results in at least one base of the complementary strand being without a base pair. A mismatch may be present in either the sense strand, or antisense strand, or both strands, of an siRNA. If more than one mismatch is present in a duplex region, the mismatches may be immediately adjacent to each other, or they may be separated by from one to more than one nucleotide. Thus, in some embodiments, a mismatch is the presence of a base in the antisense strand of an siRNA which does not pair with the corresponding base in the complementary strand of the target siRNA. In other embodiments, a mismatch is the presence of a base in the sense strand, when present, which does not pair with the corresponding base in the antisense strand of the siRNA. In yet other embodiments, a mismatch is the presence of a base in the antisense strand that does not pair with the corresponding base in the same antisense strand in a foldback hairpin siRNA.

The terms “nucleotide” and “base” are used interchangeably when used in reference to a nucleic acid sequence.

The term “RNA interference” or “RNAi” refers to the silencing or decreasing of gene expression by siRNAs. It is the process of sequence-specific, post-transcriptional gene silencing in animals and plants, initiated by siRNA that is homologous in its duplex region to the sequence of the silenced gene. The gene may be endogenous or exogenous to the organism, present integrated into a chromosome or present in a transfection vector that is not integrated into the genome. The expression of the gene is either completely or partially inhibited. RNAi may also be considered to inhibit the function of a target RNA; the function of the target RNA may be complete or partial.

The term “overexpression” refers to the production of a gene product in transgenic organisms that exceeds levels of production in normal or non-transformed organisms. As used herein, the term “altered levels” refers to the production of gene product(s) in transgenic organisms in amounts or proportions that differ from that of normal or non-transformed organisms. The terms “overexpression” and “overexpressing” and grammatical equivalents, are used in reference to levels of mRNA to indicate a level of expression approximately 3-fold higher than that typically observed in a given tissue in a control or non-transgenic animal. Levels of mRNA are measured using any of a number of techniques known to those skilled in the art including, but not limited to Northern blot analysis (See, Example 10, for a protocol for performing Northern blot analysis). Appropriate controls are included on the Northern blot to control for differences in the amount of RNA loaded from each tissue analyzed (e.g., the amount of 28S rRNA, an abundant RNA transcript present at essentially the same amount in all tissues, present in each sample can be used as a means of normalizing or standardizing the RAD50 mRNA-specific signal observed on Northern blots).

The terms “Western blot analysis” and “Western blot” and “Western” refers to the analysis of protein(s) (or polypeptides) immobilized onto a support such as nitrocellulose or a membrane. A mixture comprising at least one protein is first separated on an acrylamide gel, and the separated proteins are then transferred from the gel to a solid support, such as nitrocellulose or a nylon membrane. The immobilized proteins are exposed to at least one antibody with reactivity against at least one antigen of interest. The bound antibodies may be detected by various methods, including the use of radiolabeled antibodies.

The term “isolated” when used in relation to a nucleic acid, as in “an isolated oligonucleotide” refers to a nucleic acid sequence that is identified and separated from at least one contaminant nucleic acid with which it is ordinarily associated in its natural source. Isolated nucleic acid is present in a form or setting that is different from that in which it is found in nature. In contrast, non-isolated nucleic acids, such as DNA and RNA, are found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNA s which encode a multitude of proteins. However, isolated nucleic acid encoding a particular protein includes, by way of example, such nucleic acid in cells ordinarily expressing the protein, where the nucleic acid is in a chromosomal location different from that of natural cells, or is otherwise flanked by a different nucleic acid sequence than that found in nature. The isolated nucleic acid or oligonucleotide may be present in single-stranded or double-stranded form. When an isolated nucleic acid or oligonucleotide is to be utilized to express a protein, the oligonucleotide will contain at a minimum the sense or coding strand (i.e., the oligonucleotide may single-stranded), but may contain both the sense and anti-sense strands (i.e., the oligonucleotide may be double-stranded).

The term “purified” refers to molecules, either nucleic or amino acid sequences, that are removed from their natural environment, isolated or separated. An “isolated nucleic acid sequence” is therefore a purified nucleic acid sequence. “Substantially purified” molecules are at least 60% free, preferably at least 75% free, and more preferably at least 90% free from other components with which they are naturally associated. As used herein, the term “purified” or “to purify” also refers to the removal of contaminants from a sample. The removal of contaminating proteins results in an increase in the percent of polypeptide of interest in the sample. In another example, recombinant polypeptides are expressed in plant, bacterial, yeast, or mammalian host cells and the polypeptides are purified by the removal of host cell proteins; the percent of recombinant polypeptides is thereby increased in the sample.

The term “sample” in the present specification and claims is used in its broadest sense. On the one hand it is meant to include a specimen, culture or cell (e.g., MCF-7 cells). On the other hand, it is meant to include both biological and environmental samples.

### DETAILED DESCRIPTION OF THE INVENTION

Recently the field of reverse genetic analysis, or gene silencing, has been revolutionized by the discovery of potent, sequence-specific inactivation of gene function, which can be induced by double-stranded RNA (dsRNA). This mechanism of gene silencing is termed RNA interference (RNAi), and it has become a powerful and widely used tool for the analysis of gene function in invertebrates and plants (reviewed in Sharp, P. A. (2001) Genes Dev 15, 485-90). Introduction of double-stranded RNA (dsRNA) into the cells of these organisms leads to the sequence-specific destruction of endogenous RNAs, when one of the strands of the dsRNA corresponds to or is complementary to an endogenous RNA. The result is inhibition of the expression of the endogenous RNA. Endogenous RNA can thus be targeted for inhibition, by selecting dsRNA of which one strand is complementary to the sense strand of an endogenous RNA. During RNAi, long dsRNA molecules are processed into 19-23 nucleotide (nt) RNAs known as short-interfering RNAs (siRNAs) that serve as guides for enzymatic cleavage of complementary RNAs (Elbashir, S. M. et al. (2001) Genes Dev 15, 188-2000; Parrish, S. et al. (2000) Mol Cell 6, 1077-87; Nykanen, A. et al. (2001) Cell 107, 309-21; Elbashir, S. M. et al. (2001) Embo J 20, 6877-88; Hammond, S. M. et al. (2000) Nature 404, 293-6; Zamore, P. D. et al. (2000) Cell 101, 25-33; Bass, B. L. (2001) Nature 411, 428-9; and Yang, D. et al. (2000) Curr Biol 10, 1191-200). In addition, siRNAs can function as primers for an RNA-dependent RNA polymerase, leading to the synthesis of additional dsRNA, which in turn is processed into siRNAs to amplify the effects of the original siRNAs (Sijen, T. et al. (2001) Cell 107, 465-76; and Lipardi, C. et al. (2001) Cell 107, 297-307). Although the overall process of siRNA inhibition has been characterized, the specific enzymes that mediate siRNA function remain to be identified.

In mammalian cells, dsRNA is processed into siRNAs (Elbashir, S. M. et al. (2001) Nature 411, 494-8; Billy, E. et al. (2001) Proc Natl Acad Sci USA 98, 14428-33; and Yang, S. et al. (2001) Mol Cell Biol 21, 7807-16), but RNAi was not successful in most cell types due to nonspecific responses elicited by dsRNA molecules longer than about 30 nt (Robertson, H. D. & Mathews, M. B. (1996) Biochimie 78, 909-14). However, Tuschl and coworkers recently made the remarkable observation that transfection of synthetic 21-nt siRNA duplexes into mammalian cells effectively inhibits endogenous genes in a sequence specific manner (Elbashir, S. M. et al. (2001) Nature 411, 494-8; and Harborth, J. et al. (2001) J Cell Sci 114, 4557-65). These siRNA duplexes are too short to trigger the nonspecific dsRNA responses, but they still trigger destruction of complementary RNA sequences (Hutvagner, G. et al. (2001) Science 293, 834-8).

Accordingly, the present invention relates to compositions comprising siRNA. In particular, the present invention provides siRNA comprising nucleotide sequence that is complementary to bcl-xL mRNA sequence and that is not complementary to bcl-xS mRNA sequence, recombinant nucleic acid comprising a vector and nucleic acid sequence for expressing bcl-xL siRNA, pharmaceutical compositions comprising bcl-xL siRNA, kits comprising such compositions, and methods of using the same in research and/or therapeutic applications.

The present invention discloses siRNA, wherein the siRNA comprises a sense RNA strand and an antisense RNA strand, wherein the sense and the antisense RNA strands form an RNA duplex (e.g., dsRNA), and wherein the duplex comprises a complementary nucleotide sequence that is complementary to at least a part of bcl-xL mRNA sequence (e.g., SEQ ID NO: 1) or equivalent sequences but not complementary to bcl-xS mRNA sequence (e.g., SEQ ID NO: 2) or equivalent sequences, and wherein the complementary nucleotide sequence is less than 25 nucleotides in length, as well as compositions and methods for inhibiting the expression of a bcl-xL in a cell using the dsRNA. The present invention also discloses compositions and methods for modifying levels of bcl-xL without affecting levels of bcl-xS, inducing apoptosis in cells, and sensitizing a host or host cells to chemotherapy. The present invention also discloses compositions and methods for treating diseases in organisms caused by the aberrant expression of bcl-xL using dsRNA. The siRNA/dsRNA of the present invention directs the sequence-specific degradation of bcl-xL mRNA, without degrading bcl-xS mRNA, through a process known as RNA interference (RNAi). The process occurs in a wide variety of organisms, including mammals and other vertebrates.

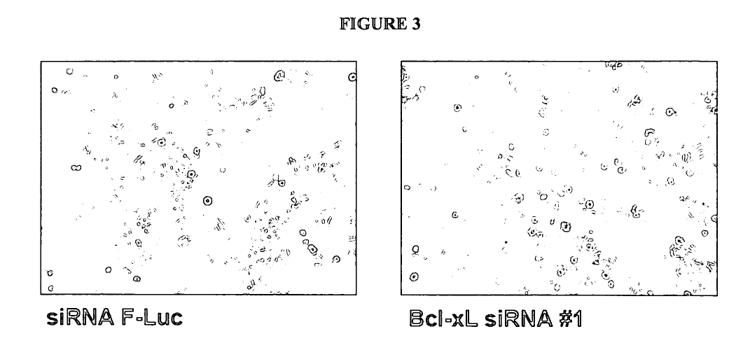

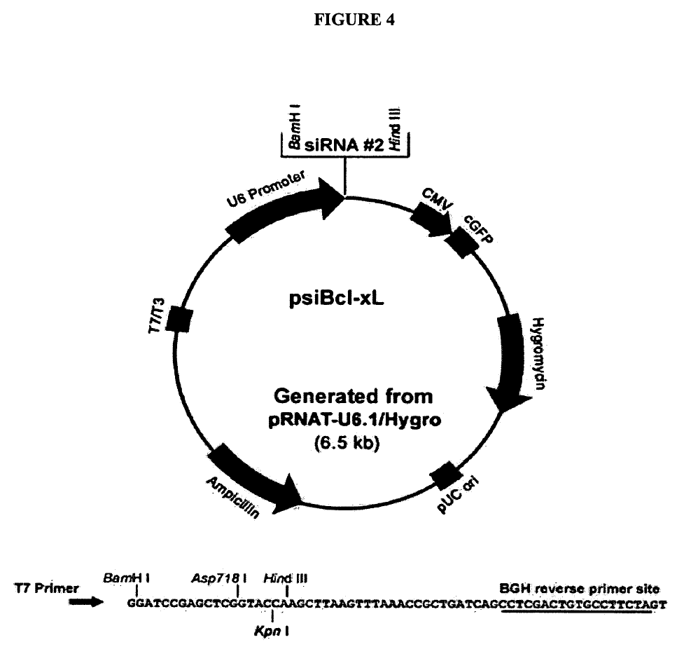

Compositions of the present invention comprise dsRNA that hybridizes to bcl-xL mRNA but that does not hybridize to bcl-xS mRNA. The dsRNA of the invention comprises an RNA strand comprising a complementary nucleotide sequence having a region which is less than 25 nucleotides in length and is complementary to at least a portion of bcl-xL RNA transcripts. The use of these dsRNAs enables the targeted degradation of bcl-xL mRNA, implicated in uncontrolled cell and tissue growth, but not bcl-xS mRNA. Very low dosages of these dsRNA can specifically and efficiently mediate RNAi, resulting in significant inhibition of expression of bcl-xL and unaffecting the expression of bcl-xS (See, e.g., Example 4, FIGS. 3-4). The dsRNA of the present invention not only reduce the level of bcl-xL, they also decrease the tumorigenecity of known and well characterized tumor cells (See, e.g., Example 6), and sensitize tumor cells to treatment with chemotherapeutic agents (See, e.g., Example 5). Thus, the present invention encompasses these dsRNAs and compositions comprising dsRNA and their use for specifically silencing bcl-xL whose protein product either inhibits or prevents apoptosis in tumor cells. Moreover, the dsRNAs of the invention have no apparent effect on neighboring normal cells. Thus, the methods and compositions of the present invention comprising these dsRNAs are useful for treating cellular proliferative and/or differentiation disorders, such as cancer.

The following detailed description discloses how to make and use the siRNA/dsRNA of the present invention and compositions containing dsRNA to inhibit the expression of bcl-xL, as well as compositions and methods for treating diseases and disorders caused by the expression of bcl-xL. The pharmaceutical compositions of the present invention comprise a dsRNA having an RNA strand comprising a complementary nucleotide sequence that is less than 25 nucleotides in length and is complementary to at least a portion of a bcl-xL RNA transcript of bcl-xL, together with a pharmaceutically acceptable carrier. The pharmaceutical composition may comprise a combination of dsRNAs having regions complementary to a plurality of sites within bcl-xL that are not complementary to bcl-xS (e.g., a pharmaceutical composition that comprises siRNA #1 and siRNA #2 of Example 1). The compositions of the present invention can be used in combination with other known therapeutic agents (e.g., chemotherapeutic agents).

Accordingly, certain aspects of the present invention relate to pharmaceutical compositions comprising the dsRNA of the present invention, or vectors containing the same, together with a pharmaceutically acceptable carrier, methods of using the compositions to inhibit expression of bcl-xL without affecting expression of bcl-xS, and methods of using the pharmaceutical compositions to treat diseases caused by expression of bcl-xL.

I. Pharmaceutical Compositions Comprising dsRNA

a. Double-Stranded Ribonucleic Acid (dsRNA)

In one embodiment, the invention relates to a RNA duplex/double-stranded ribonucleic acid (dsRNA) having a nucleotide sequence which is substantially identical to at least a portion of bcl-xL. The dsRNA comprises two RNA strands that are sufficiently complementary to hybridize to form the duplex structure. One strand of the dsRNA comprises the nucleotide sequence that is substantially identical to a portion of the target gene (the “sense” strand), and the other strand (the “complementary” or “antisense” strand) comprises a complementary nucleotide sequence that is complementary to an RNA transcript of the target gene. The complementary region is between 19 and 24, preferably between 21 and 23, and most preferably 22 nucleotides in length. The dsRNA is less than 30 nucleotides, preferably less than 25 nucleotides, and most preferably between 21 and 24 nucleotides in length. The dsRNA can be synthesized by standard methods known in the art, e.g., by use of an automated DNA synthesizer, such as are commercially available from Biosearch, Applied Biosystems, Inc, etc. In specific embodiments, the complementary (e.g., antisense) RNA strand of the dsRNA is encoded by the sequence set forth in SEQ ID NOS: 3-9 (See, e.g., Example 1, underlined sequence) and the second (sense) RNA strand is encoded by the sequence set forth in SEQ ID NOS: 3-9 (See, e.g., Example 1, italicized sequence).

In one embodiment, at least one end of the dsRNA has a single-stranded nucleotide overhang of 1 to 7, preferably 1 or 2 nucleotides. dsRNAs having at least one nucleotide overhang have unexpectedly superior inhibitory properties than their blunt-ended counterparts. dsRNA having only one overhang has proven particularly stable and effective in vivo, as well as in a variety of cells, cell culture mediums, blood, and serum. Preferably, the single-stranded overhang is located at the 3′-terminal end of the complementary (antisense) RNA strand or, alternatively, at the 3′-terminal end of the second (sense) strand. The dsRNA may also have a blunt end, preferably located at the 5′-end of the complementary (antisense) strand. Such dsRNAs have improved stability and inhibitory activity, thus allowing administration at low dosages, i.e., less than 5 mg/kg body weight of the recipient per day. Preferably, the complementary strand of the dsRNA has a nucleotide overhang at the 3′-end, and the 5′-end is blunt. In another embodiment, one or more of the nucleotides in the overhang is replaced with a nucleoside thiophosphate.

In yet another embodiment, the dsRNA is chemically modified for improved stability, i.e., enhanced resistance to degradation and/or strand dissociation. In this embodiment, the integrity of the duplex structure is strengthened by at least one, and preferably two, chemical linkages. Chemical linking may be achieved by any of a variety of well-known techniques, for example by introducing covalent, ionic or hydrogen bonds; hydrophobic interactions, van der Waals or stacking interactions; by means of metal-ion coordination, or through use of purine analogues. In one embodiment, the linker is a hexa-ethylene glycol linker. In this case, the dsRNAs are produced by solid phase synthesis and the hexa-ethylene glycol linker is incorporated according to standard methods (See, e.g., Williams, D. J., and K. B. Hall, Biochem. (1996) 35:14665-14670). In a preferred embodiment, the 5′-end of the complementary (antisense) RNA strand and the 3′-end of the second (sense) RNA strand are chemically linked via a hexa-ethylene glycol linker. In yet another preferred embodiment, the dsRNA is linked via a single stranded nucleic acid forming a loop or hairpin structure.

In one embodiment, the invention relates to a pharmaceutical composition comprising a dsRNA, as described in the preceding section, and a pharmaceutically acceptable carrier, as described below. The pharmaceutical composition comprising the dsRNA is useful for treating a disease or disorder associated with the expression or activity of bcl-xL (e.g., cancer).

In another embodiment, the invention relates to a pharmaceutical composition comprising at least two dsRNAs, designed to target different regions of bcl-xL, but that does not target bcl-xS, and a pharmaceutically acceptable carrier. A composition comprising a plurality of dsRNA that target multiple regions of bcl-xL may provide improved efficiency of treatment as compared to compositions comprising only a single dsRNA. In addition, a composition may be generated that includes the dsRNA of the present invention and other known dsRNAs that target other anti-apoptotic genes (e.g., bcl-2). For example, one dsRNA may have a nucleotide sequence which is substantially identical to at least a portion bcl-xL that does not bind to bcl-xS; additional dsRNAs are prepared, each of which has a nucleotide sequence that is substantially identical to a portion of a different anti-apoptotic gene (e.g., bcl-2). The multiple dsRNAs may be combined in the same pharmaceutical composition, or formulated separately. If formulated individually, the compositions containing the separate dsRNAs may comprise the same or different carriers, and may be administered using the same or different routes of administration. Moreover, the pharmaceutical compositions comprising the individual dsRNAs may be administered substantially simultaneously, sequentially, or at preset intervals throughout the day or treatment period. The present invention contemplates use of the dsRNAs of the present invention in combinatorial use with dsRNA for any gene or combination of genes that have an inhibitory or preventive effect on apoptosis.

b. Recombinant Nucleic Acids Comprising a Vector and Nucleic Acid Sequence for Expressing dsRNA

In some embodiments, the dsRNA of the invention can be obtained using a number of techniques known to those of skill in the art. For example, the dsRNA can be chemically synthesized or recombinantly produced using methods known in the art, such as the Drosophila in vitro system described in U.S. published application 2002/0086356 of Tuschl et al., the entire disclosure of which is herein incorporated by reference.

In some embodiments, the dsRNA of the invention are chemically synthesized using appropriately protected ribonucleoside phosphoramidites and a conventional DNA/RNA synthesizer. The dsRNA can be synthesized as two separate, complementary RNA molecules, or as a single RNA molecule with two complementary regions. Commercial suppliers of synthetic RNA molecules or synthesis reagents include Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA) and Cruachem (Glasgow, UK).

In preferred embodiments, dsRNA is expressed from recombinant circular or linear DNA plasmids using any suitable promoter. Suitable promoters for expressing dsRNA of the invention from a plasmid include, for example, the U6 or H1 RNA pol III promoter sequences and the cytomegalovirus promoter. Selection of other suitable promoters is within the skill in the art. The recombinant plasmids of the invention can also comprise inducible or regulatable promoters for expression of the dsRNA in a particular tissue or in a particular intracellular environment.

dsRNA of the invention can be expressed from a recombinant plasmid either as two separate, complementary RNA molecules, or as a single RNA molecule with two complementary regions.

Selection of plasmids suitable for expressing dsRNA of the invention, methods for inserting nucleic acid sequences for expressing the dsRNA into the plasmid, and methods of delivering the recombinant plasmid to the cells of interest are within the skill in the art (See, e.g., Tuschl, T. (2002), Nat. Biotechnol, 20: 446-448; Brummelkamp T R et al. (2002), Science 296: 550-553; Miyagishi M et al. (2002), Nat. Biotechnol. 20: 497-500; Paddison P J et al. (2002), Genes Dev. 16: 948-958; Lee N S et al. (2002), Nat. Biotechnol. 20: 500-505; and Paul C P et al. (2002), Nat. Biotechnol. 20: 505-508, the entire disclosures of which are herein incorporated by reference).

In one embodiment, a plasmid expressing a dsRNA of the invention comprises a sense RNA strand coding sequence in operable connection with a polyT termination sequence under the control of a human U6 RNA promoter, and an antisense RNA strand coding sequence in operable connection with a polyT termination sequence under the control of a human U6 RNA promoter. Such a plasmid can be used in producing an recombinant adeno-associated viral vector for expressing a dsRNA of the invention.

The dsRNA expressed from recombinant plasmids can either be isolated from cultured cell expression systems by standard techniques, or can be expressed intracellularly. Any viral vector capable of accepting the coding sequences for the dsRNA molecule(s) to be expressed can be used, for example vectors derived from adenovirus (AV); adeno-associated virus (AAV); retroviruses (e.g, lentiviruses (LV), Rhabdoviruses, murine leukemia virus); herpes virus, and the like. The tropism of the viral vectors can be modified by pseudotyping the vectors with envelope proteins or other surface antigens from other viruses, or by substituting different viral capsid proteins, as appropriate.

For example, lentiviral vectors of the invention can be pseudotyped with surface proteins from vesicular stomatitis virus (VSV), rabies, Ebola, Mokola, and the like. AAV vectors of the invention can be made to target different cells by engineering the vectors to express different capsid protein serotypes. For example, an AAV vector expressing a serotype 2 capsid on a serotype 2 genome is called AAV 2/2. This serotype 2 capsid gene in the AAV 2/2 vector can be replaced by a serotype 5 capsid gene to produce an AAV 2/5 vector. Techniques for constructing AAV vectors which express different capsid protein serotypes are within the skill in the art; see, e.g., Rabinowitz J E et al. (2002), J Virol 76:791-801, the entire disclosure of which is herein incorporated by reference.

Selection of recombinant viral vectors suitable for use in the invention, methods for inserting nucleic acid sequences for expressing the dsRNA into the vector, and methods of delivering the viral vector to the cells of interest are within the skill in the art. (See, e.g., Dornburg R (1995), Gene Therap. 2: 301-310; Eglitis M A (1988), Biotechniques 6: 608-614; Miller A D (1990), Hum Gene Therap. 1: 5-14; Anderson W F (1998), Nature 392: 25-30; and GenScript siRNA Construct Builder, www.GenScript.com) the entire disclosures of which are herein incorporated by reference.

Preferred viral vectors are those derived from lentivirus, AV or AAV. In a particularly preferred embodiment, the siRNA of the invention is expressed from a single sequence inserted into a plasmid (e.g., pRNAT-U6.1/Hygro from GenScript) (See, e.g., Examples 1 and 3).

Suitable AV vectors for expressing the dsRNA of the invention, a method for constructing the recombinant AV vector, and a method for delivering the vector into target cells, are described in Xia H et al. (2002), Nat. Biotech. 20: 1006-1010. Suitable AAV vectors for expressing the siRNA of the invention, methods for constructing the recombinant AAV vector, and methods for delivering the vectors into target cells are described in Samulski R et al. (1987), J. Virol. 61: 3096-3101; Fisher K J et al. (1996), J. Virol., 70: 520-532; Samulski Ret al. (1989), J. Virol. 63: 3822-3826; U.S. Pat. No. 5,252,479; U.S. Pat. No. 5,139,941; International Patent Application No. WO 94/13788; and International Patent Application No. WO 93/24641, the entire disclosures of which are herein incorporated by reference.

II. Administration of dsRNA

The pharmaceutical, diagnostic and research compositions of the present invention are administered in dosages sufficient to inhibit expression of the target gene. Compositions comprising the dsRNA of the invention can be administered at low dosages. For example, a dosage of 5 mg dsRNA per kilogram body weight of recipient per day is contemplated to be sufficient to inhibit or completely suppress expression of the bcl-xL gene, although other dosages (e.g., greater than, or more preferably less than 5 mg dsRNA per kilogram body weight of recipient) may be used.

In general, a suitable dose of dsRNA will be in the range of 0.01 to 5.0 milligrams per kilogram body weight of the recipient per day, preferably in the range of 0.1 to 200 micrograms per kilogram body weight per day, more preferably in the range of 0.1 to 100 micrograms per kilogram body weight per day, even more preferably in the range of 1.0 to 50 micrograms per kilogram body weight per day, and most preferably in the range of 1.0 to 25 micrograms per kilogram body weight per day. The pharmaceutical composition may be administered once daily, or the dsRNA may be administered as two, three, four, five, six or more sub-doses at appropriate intervals throughout the day. In that case, the dsRNA contained in each sub-dose must be correspondingly smaller in order to achieve the total daily dosage. The dosage unit can also be compounded for delivery over several days, e.g., using a conventional sustained release formulation which provides sustained release of the dsRNA over a several day period. Sustained release formulations are well known in the art. In this embodiment, the dosage unit contains a corresponding multiple of the daily dose.

The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a composition can include a single treatment or a series of treatments. Estimates of effective dosages and in vivo half-lives for the individual dsRNAs encompassed by the invention can be made using conventional methodologies or on the basis of in vivo testing using an appropriate animal model, as described elsewhere herein.

Advances in mouse genetics have generated a number of mouse models for the study of various human diseases. For example, mouse models are available for hematopoictic malignancies such as leukemias, lymphomas and acute myclogenous leukemia. The MMHCC (Mouse models of Human Cancer Consortium) web page (emice.nci.nih.gov), sponsored by the National Cancer Institute, provides disease-site-specific compendium of known cancer models, and has links to the searchable Cancer Models Database (cancermodels.nci.nih.gov), as well as the NCI-MMHCC mouse repository. Examples of the genetic tools that are currently available for the modeling of leukemia and lymphomas in mice, and which are useful in practicing the present invention, are described in the following references: Maru, Y., Int. J. Hematol. (2001) 73:308-322; Pandolfi, P. P., Oncogene (2001) 20:5726-5735; Pollock, J. L., et al., Curr. Opin. Hematol. (2001) 8:206-211; Rego, E. M., et al., Semin. in Hemat. (2001) 38:4-70; Shannon, K. M., et al. (2001) Modeling myeloid leukemia tumors suppressor gene inactivation in the mouse, Semin. Cancer Biol. 11, 191-200; Van Etten, R. A., (2001) Curr. Opin. Hematol. 8, 224-230; Wong, S., et al. (2001) Oncogene 20, 5644-5659; Phillips J A., Cancer Res. (2000) 52(2):437-43; Harris, A. W., et al, J. Exp. Med. (1988) 167(2):353-71; Zeng X X et al., Blood. (1988) 92(10):3529-36; Eriksson, B., et al., Exp. Hematol. (1999) 27(4):682-8; and Kovalchuk, A. et al., J. Exp. Med. (2000) 192(8): 1183-90. Mouse repositories can also be found at: The Jackson Laboratory, Charles River Laboratories, Taconic, Harlan, Mutant Mouse Regional Resource Centers (MMRRC) National Network and at the European Mouse Mutant Archive. Such models may be used for in vivo testing of dsRNA, as well as for determining a therapeutically effective dose.

The pharmaceutical compositions encompassed by the invention may be administered by any means known in the art including, but not limited to oral or parenteral routes, including intravenous, intramuscular, intraperitoneal, subcutaneous, transdermal, airway (aerosol), rectal, vaginal and topical (including buccal and sublingual) administration. In preferred embodiments, the pharmaceutical compositions are administered by intravenous or intraparenteral infusion or injection.

For oral administration, the dsRNAs useful in the invention will generally be provided in the form of tablets or capsules, as a powder or granules, or as an aqueous solution or suspension.

Tablets for oral use may include the active ingredients mixed with pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract.

Capsules for oral use include hard gelatin capsules in which the active ingredient is mixed with a solid diluent, and soft gelatin capsules wherein the active ingredients is mixed with water or an oil such as peanut oil, liquid paraffin or olive oil.

For intramuscular, intraperitoneal, subcutaneous and intravenous use, the pharmaceutical compositions of the invention will generally be provided in sterile aqueous solutions or suspensions, buffered to an appropriate pH and isotonicity. Suitable aqueous vehicles include Ringer's solution and isotonic sodium chloride. In a preferred embodiment, the carrier consists exclusively of an aqueous buffer. In this context, “exclusively” means no auxiliary agents or encapsulating substances are present that might affect or mediate uptake of dsRNA in the cells that express bcl-xL. Such substances include, for example, micellar structures, such as liposomes or capsids, as described below. Compositions containing only naked dsRNA and a physiologically acceptable solvent are taken up by cells, where the dsRNA effectively inhibits expression of bcl-xL. Although microinjection, lipofection, viruses, viroids, capsids, capsoids, or other auxiliary agents are required to introduce dsRNA into cell cultures, these methods and agents are not necessary for uptake of dsRNA in vivo. Aqueous suspensions according to the invention may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

The pharmaceutical compositions useful according to the invention also include encapsulated formulations to protect the dsRNA against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811; PCT publication WO 91/06309; and European patent publication EP-A-43075, which are incorporated by reference herein.

In one embodiment, the encapsulated formulation comprises a viral coat protein. In this embodiment, the dsRNA may be bound to, associated with, or enclosed by at least one viral coat protein. The viral coat protein may be derived from or associated with a virus, such as a polyoma virus, or it may be partially or entirely artificial. For example, the coat protein may be a Virus Protein 1 and/or Virus Protein 2 of the polyoma virus, or a derivative thereof.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred.

The data obtained from cell culture assays and animal studies can be used in a range of dosage formulations for use in humans. The dosage of compositions of the invention lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range of the compound or, when appropriate, of the polypeptide product of a target sequence (e.g., achieving a decreased concentration of the polypeptide) that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

In addition to their administration individually or as a plurality, as discussed above, the dsRNAs useful according to the invention can be administered in combination with other known agents effective in treatment of diseases. In any event, the administering physician can adjust the amount and timing of dsRNA administration on the basis of results observed using standard measures of efficacy known in the art or described herein. For oral administration, the dsRNAs useful in the invention will generally be provided in the form of tablets or capsules, as a powder or granules, or as an aqueous solution or suspension.

In some embodiments, systemic gene delivery system is used to deliver recombinant nucleic acid comprising a vector and nucleic acid sequence for expressing a dsRNA comprising a sense RNA strand and an antisense RNA strand, wherein the sense and the antisense RNA strands form an RNA duplex, and wherein the duplex comprises a complementary nucleotide sequence that is complementary to at least a part of bcl-xL mRNA sequence but not complementary to bcl-xS mRNA sequence, and wherein said complementary nucleotide sequence is less than 25 nucleotides in length. In preferred embodiments, the gene delivery system is the system described in U.S. Pat. No. 6,749,863 (herein incorporated by reference in its entirety) which provides targeted liposome gene delivery.

In some embodiments, the dsRNA can be delivered directly to a site of tumorigenesis. For example, dsRNA can be targeted directly to a site of tumorigenesis using a tumor-targeted liposomal dsRNA oligonucleotide complex using an anti-transferrin receptor single-chain antibody fragment (TfRscFv) as the targeting entity.

III. Methods for Treating Diseases Caused by Expression of bcl-xL.

In one embodiment, the invention relates to a method for treating a subject having a disease or at risk of developing a disease caused by the expression of bcl-xL. In this embodiment, the dsRNA can act as novel therapeutic agents for controlling one or more of cellular proliferative and/or differentiative disorders. The method comprises administering a pharmaceutical composition of the invention to the patient (e.g., human), such that expression of bcl-xL, but not bcl-xS, is silenced.

The dsRNA of the present invention can be uses to prevent or treat a disease caused by the aberrant expression of bcl-xL. In preferred embodiments, the compositions of the present invention are used to treat cancer and other types of disease caused by expression of bcl-xL.

IV. Methods of Sensitizing Cells to Chemotherapy

In preferred embodiments, the present invention provides methods of sensitizing a subject to chemotherapeuitc agents comprising administering to the subject an effective amount of a pharmaceutical composition comprising a dsRNA comprising a sense RNA strand and an antisense RNA strand, wherein the sense and the antisense RNA strands form an RNA duplex, and wherein the duplex comprises a complementary nucleotide sequence that is complementary to at least a part of bcl-xL mRNA sequence but not complementary to bcl-xS mRNA sequence, and wherein the complementary nucleotide sequence is less than 25 nucleotides in length. In some embodiments, the dsRNA of the present invention is administered to a subject and independently sensitizes the subject to chemotherapeutic agents (See, e.g., Example 5).

In some embodiments, to enhance the sensitizing, the compositions of the present invention are used in combination, as described above, with dsRNA to inhibit the expression of the multi-drug resistance 1 gene (“MDR1”), or other genes that render cells more responsive to treatment. “Multi-drug resistance” (MDR) broadly refers to a pattern of resistance to a variety of chemotherapeutic drugs with unrelated chemical structures and different mechanisms of action. Although the etiology of MDR is multifactorial, the overexpression of P-glycoprotein (Pgp), a membrane protein that mediates the transport of MDR drugs, remains the most common alteration underlying MDR in laboratory models (Childs, S., Imp. Adv. Oncol. (1994) 21-36). Moreover, expression of Pgp has been linked to the development of MDR in human cancer, particularly in the leukemias, lymphomas, multiple myeloma, neuroblastoma, and soft tissue sarcoma (Fan., D., et al., Reversal of Multidrug Resistance in Cancer, ed. Kellen, J. A. (CRC, Boca Raton, Fla.), pp. 93-125). Recent studies showed that tumor cells expressing MDR-associated protein (MRP) (Cole, S. P. C., et al., Science (1992) 258:1650-1654) and lung resistance protein (LRP) (Scheffer, G. L., et al., Nat. Med. (1995)1:578-582) and mutation of DNA topoisomerase II (Beck, W. T., J. Natl. Cancer Inst. (1989) 81:1683-1685) also may render MDR.

The compositions and methods of the present invention can be used in combination with any oncolytic agent that is routinely used in a cancer therapy. For example, the U.S. Food and Drug Administration maintains a formulary of oncolytic agents approved for use in the United States. International counterpart agencies to the U.S.F.D.A. maintain similar formularies. Table 2 provides a list of exemplary antineoplastic agents approved for use in the U.S. Those skilled in the art will appreciate that the “product labels” required on all U.S. approved chemotherapeutics describe approved indications, dosing information, toxicity data, and the like, for the exemplary agents.

TABLE 6

| | | |
|---|---|---|
| Aldesleukin | Proleukin | Chiron Corp., |
| (des-alanyl-1, serine-125 human interleukin-2) | | Emeryville, CA |
| Alemtuzumab | Campath | Millennium and |
| (IgG1κ anti CD52 antibody) | | ILEX Partners, LP, |
| | | Cambridge, MA |
| Alitretinoin | Panretin | Ligand |
| (9-cis-retinoic acid) | | Pharmaceuticals, Inc., |
| | | San Diego CA |
| Allopurinol | Zyloprim | GlaxoSmithKline, |
| (1,5-dihydro-4H-pyrazolo(3,4-d)pyrimidin-4-one | | Research Triangle |
| monosodium salt) | | Park, NC |
| Altretamine | Hexalen | US Bioscience, West |
| (N,N,N′,N′,N″,N″,-hexamethyl-1,3,5-triazine-2,4, | | Conshohocken, PA |
| 6-triamine) | | |
| Amifostine | Ethyol | US Bioscience |
| (ethanethiol, 2-((3-aminopropyl)amino)-, | | |
| dihydrogen phosphate (ester)) | | |
| Anastrozole | Arimidex | AstraZeneca |
| (1,3-Benzenediacetonitrile, a,a,a′,a′-tetramethyl- | | Pharmaceuticals, LP, |
| 5-(1H-1,2,4-triazol-1-ylmethyl)) | | Wilmington, DE |
| Arsenic trioxide | Trisenox | Cell Therapeutic, |
| | | Inc., Seattle, WA |
| Asparaginase | Elspar | Merck & Co., Inc., |
| (L-asparagine amidohydrolase, type EC-2) | | Whitehouse Station, |
| | | NJ |
| BCG Live | TICE BCG | Organon Teknika, |
| (lyophilized preparation of an attenuated strain of | | Corp., Durham, NC |
| Mycobacterium bovis (Bacillus Calmette-Gukin | | |
| (BCG), substrain Montreal) | | |
| bexarotene capsules | Targretin | Ligand |
| (4-(1-(5,6,7,8-tetrahydro-3,5,5,8,8-pentamethyl-2- | | Pharmaceuticals |
| napthalenyl) ethenyl) benzoic acid) | | |
| bexarotene gel | Targretin | Ligand |
| | | Pharmaceuticals |
| Bleomycin | Blenoxane | Bristol-Myers Squibb |
| (cytotoxic glycopeptide antibiotics produced by | | Co., NY, NY |
| Streptomyces verticillus; bleomycin A₂ and | | |
| bleomycin B₂) | | |
| Capecitabine | Xeloda | Roche |
| (5′-deoxy-5-fluoro-N-((pentyloxy)carbonyl)- | | |
| cytidine) | | |
| Carboplatin | Paraplatin | Bristol-Myers Squibb |
| (platinum, diammine (1,1- | | |
| cyclobutanedicarboxylato(2−)-0,0′)-, (SP-4-2)) | | |
| Carmustine | BCNU, BiCNU | Bristol-Myers Squibb |
| (1,3-bis(2-chloroethyl)-1-nitrosourea) | | |
| Carmustine with Polifeprosan 20 Implant | Gliadel Wafer | Guilford |
| | | Pharmaceuticals, Inc., |
| | | Baltimore, MD |
| Celecoxib | Celebrex | Searle |
| (as 4-(5-(4-methylphenyl)-3-(trifluoromethyl)- | | Pharmaceuticals, |
| 1H-pyrazol-1-yl) | | England |
| benzenesulfonamide) | | |
| Chlorambucil | Leukeran | GlaxoSmithKline |
| (4-(bis(2chlorethyl)amino)benzenebutanoic acid) | | |
| Cisplatin | Platinol | Bristol-Myers Squibb |
| (PtCl₂H₆N₂) | | |
| Cladribine | Leustatin, 2-CdA | R. W. Johnson |
| (2-chloro-2′-deoxy-b-D-adenosine) | | Pharmaceutical |
| | | Research Institute, |
| | | Raritan, NJ |
| Cyclophosphamide | Cytoxan, Neosar | Bristol-Myers Squibb |
| (2-(bis(2-chloroethyl)amino) tetrahydro-2H-13,2- | | |
| oxazaphosphorine 2-oxide monohydrate) | | |
| Cytarabine | Cytosar-U | Pharmacia & Upjohn |
| (1-b-D-Arabinofuranosylcytosine, C₉H₁₃N₃O₅) | | Company |
| cytarabine liposomal | DepoCyt | Skye |
| | | Pharmaceuticals, Inc., |
| | | San Diego, CA |
| Dacarbazine | DTIC-Dome | Bayer AG, |
| (5-(3,3-dimethyl-1-triazeno)-imidazole-4- | | Leverkusen, |
| carboxamide (DTIC)) | | Germany |
| Dactinomycin, actinomycin D | Cosmegen | Merck |
| (actinomycin produced by Streptomyces parvullus, | | |
| C₆₂H₈₆N₁₂O₁₆) | | |
| Darbepoetin alfa | Aranesp | Amgen, Inc., |
| (recombinant peptide) | | Thousand Oaks, CA |
| daunorubicin liposomal | DanuoXome | Nexstar |
| ((8S-cis)-8-acetyl-10-((3-amino-2,3,6-trideoxy-a- | | Pharmaceuticals, Inc., |
| L-lyxo-hexopyranosyl)oxy)-7,8,9,10-tetrahydro- | | Boulder, CO |
| 6,8,11-trihydroxy-1-methoxy-5,12- | | |
| naphthacenedione hydrochloride) | | |
| Daunorubicin HCl, daunomycin | Cerubidine | Wyeth Ayerst, |
| ((1S,3S)-3-Acetyl-1,2,3,4,6,11-hexahydro- | | Madison, NJ |
| 3,5,12-trihydroxy-10-methoxy-6,11-dioxo-1- | | |
| naphthacenyl 3-amino-2,3,6-trideoxy-(alpha)-L- | | |
| lyxo-hexopyranoside hydrochloride) | | |
| Denileukin diftitox | Ontak | Seragen, Inc., |
| (recombinant peptide) | | Hopkinton, MA |
| Dexrazoxane | Zinecard | Pharmacia & Upjohn |
| ((S)-4,4′-(1-methyl-1,2-ethanediyl)bis-2,6- | | Company |
| piperazinedione) | | |
| Docetaxel | Taxotere | Aventis |
| ((2R,3S)-N-carboxy-3-phenylisoserine, N-tert- | | Pharmaceuticals, Inc., |
| butyl ester, 13-ester with 5b-20-epoxy- | | Bridgewater, NJ |
| 12a,4,7b,10b,13a-hexahydroxytax-11-en-9-one 4- | | |
| acetate 2-benzoate, trihydrate) | | |
| Doxorubicin HCl | Adriamycin, | Pharmacia & Upjohn |
| (8S,10S)-10-((3-amino-2,3,6-trideoxy-a-L-lyxo- | Rubex | Company |
| hexopyranosyl)oxy)-8-glycolyl-7,8,9,10- | | |
| tetrahydro-6,8,11-trihydroxy-1-methoxy-5,12- | | |
| naphthacenedione hydrochloride) | | |
| doxorubicin | Adriamycin PFS | Pharmacia & Upjohn |
| | Intravenous | Company |
| | injection | |
| doxorubicin liposomal | Doxil | Sequus |
| | | Pharmaceuticals, Inc., |
| | | Menlo park, CA |
| dromostanolone propionate | Dromostanolone | Eli Lilly & Company, |
| (17b-Hydroxy-2a-methyl-5a-androstan-3-one | | Indianapolis, IN |
| propionate) | | |
| dromostanolone propionate | Masterone | Syntex, Corp., Palo |
| | injection | Alto, CA |
| Elliott′s B Solution | Elliott′s B | Orphan Medical, Inc |
| | Solution | |
| Epirubicin | Ellence | Pharmacia & Upjohn |
| ((8S-cis)-10-((3-amino-2,3,6-trideoxy-a-L- | | Company |
| arabino-hexopyranosyl)oxy)-7,8,9,10-tetrahydro- | | |
| 6,8,11-trihydroxy-8-(hydroxyacetyl)-1-methoxy- | | |
| 5,12-naphthacenedione hydrochloride) | | |
| Epoetin alfa | Epogen | Amgen, Inc |
| (recombinant peptide) | | |
| Estramustine | Emcyt | Pharmacia & Upjohn |
| (estra-1,3,5(10)-triene-3,17-diol(17(beta))-, 3- | | Company |
| (bis(2-chloroethyl)carbamate) 17-(dihydrogen | | |
| phosphate), disodium salt, monohydrate, or | | |
| estradiol 3-(bis(2-chloroethyl)carbamate) 17- | | |
| (dihydrogen phosphate), disodium salt, | | |
| monohydrate) | | |
| Etoposide phosphate | Etopophos | Bristol-Myers Squibb |
| (4′-Demethylepipodophyllotoxin 9-(4,6-O-(R)- | | |
| ethylidene-(beta)-D-glucopyranoside), 4′- | | |
| (dihydrogen phosphate)) | | |
| etoposide, VP-16 | Vepesid | Bristol-Myers Squibb |
| (4′-demethylepipodophyllotoxin 9-(4,6-0-(R)- | | |
| ethylidene-(beta)-D-glucopyranoside)) | | |
| Exemestane | Aromasin | Pharmacia & Upjohn |
| (6-methylenandrosta-1,4-diene-3,17-dione) | | Company |
| Filgrastim | Neupogen | Amgen, Inc |
| (r-metHuG-CSF) | | |
| floxuridine (intraarterial) | FUDR | Roche |
| (2′-deoxy-5-fluorouridine) | | |
| Fludarabine | Fludara | Berlex Laboratories, |
| (fluorinated nucleotide analog of the antiviral | | Inc., Cedar Knolls, |
| agent vidarabine, 9-b-D-arabinofuranosyladenine | | NJ |
| (ara-A)) | | |
| Fluorouracil, 5-FU | Adrucil | ICN Pharmaceuticals, |
| (5-fluoro-2,4(1H,3H)-pyrimidinedione) | | Inc., Humacao, |
| | | Puerto Rico |
| Fulvestrant | Faslodex | IPR Pharmaceuticals, |
| (7-alpha-(9-(4,4,5,5,5-penta fluoropentylsulphinyl) | | Guayama, Puerto |
| nonyl)estra-1,3,5-(10)-triene-3,17-beta-diol) | | Rico |
| Gemcitabine | Gemzar | Eli Lilly |
| (2′-deoxy-2′,2′-difluorocytidine | | |
| monohydrochloride (b-isomer)) | | |
| Gemtuzumab Ozogamicin | Mylotarg | Wyeth Ayerst |
| (anti-CD33 hP67.6) | | |
| Goserelin acetate | Zoladex Implant | AstraZeneca |
| (acetate salt of (D-Ser(But)⁶, Azgly¹⁰)LHRH; pyro- | | Pharmaceuticals |
| Glu-His-Trp-Ser-Tyr-D-Ser(But)-Leu-Arg-Pro- | | |
| Azgly-NH2 acetate (C₅₉H₈₄N₁₈O₁₄.(C₂H₄O₂)ₓ | | |
| Hydroxyurea | Hydrea | Bristol-Myers Squibb |
| Ibritumomab Tiuxetan | Zevalin | Biogen IDEC, Inc., |
| (immunoconjugate resulting from a thiourea | | Cambridge MA |
| covalent bond between the monoclonal antibody | | |
| Ibritumomab and the linker-chelator tiuxetan (N- | | |
| (2-bis(carboxymethyl)amino)-3-(p- | | |
| isothiocyanatophenyl)-propyl)-(N-(2- | | |
| bis(carboxymethyl)amino)-2-(methyl)- | | |
| ethyl)glycine) | | |
| Idarubicin | Idamycin | Pharmacia & Upjohn |
| (5,12-Naphthacenedione, 9-acetyl-7-((3-amino- | | Company |
| 2,3,6-trideoxy-(alpha)-L-lyxo- | | |
| hexopyranosyl)oxy)-7,8,9,10-tetrahydro-6,9,11- | | |
| trihydroxyhydrochloride, (7S-cis)) | | |
| Ifosfamide | IFEX | Bristol-Myers Squibb |
| (3-(2-chloroethyl)-2-((2- | | |
| chloroethyl)amino)tetrahydro-2H-1,3,2- | | |
| oxazaphosphorine 2-oxide) | | |
| Imatinib Mesilate | Gleevec | Novartis AG, Basel, |
| (4-((4-Methyl-1-piperazinyl)methyl)-N-(4-methyl- | | Switzerland |
| 3-((4-(3-pyridinyl)-2-pyrimidinyl)amino)- | | |
| phenyl)benzamide methanesulfonate) | | |
| Interferon alfa-2a | Roferon-A | Hoffmann-La Roche, |
| (recombinant peptide) | | Inc., Nutley, NJ |
| Interferon alfa-2b | Intron A | Schering AG, Berlin, |
| (recombinant peptide) | (Lyophilized | Germany |
| | Betaseron) | |
| Irinotecan HCl | Camptosar | Pharmacia & Upjohn |
| ((4S)-4,11-diethyl-4-hydroxy-9-((4-piperidinopiperidino) | | Company |
| carbonyloxy)-1H-pyrano(3′,4′:6,7) | | |
| indolizino(1,2-b) quinoline-3,14(4H,12H) | | |
| dione hydrochloride trihydrate) | | |
| Letrozole | Femara | Novartis |
| (4,4′-(1H-1,2,4-Triazol-1-ylmethylene) | | |
| dibenzonitrile) | | |
| Leucovorin | Wellcovorin, | Immunex, Corp., |
| (L-Glutamic acid, N(4(((2amino-5-formyl- | Leucovorin | Seattle, WA |
| 1,4,5,6,7,8 hexahydro4oxo6- | | |
| pteridinyl)methyl)amino)benzoyl), calcium salt | | |
| (1:1)) | | |
| Levamisole HCl | Ergamisol | Janssen Research |
| ((−)-(S)-2,3,5,6-tetrahydro-6-phenylimidazo (2,1- | | Foundation, |
| b) thiazole monohydrochloride C₁₁H₁₂N₂S.HCl) | | Titusville, NJ |
| Lomustine | CeeNU | Bristol-Myers Squibb |
| (1-(2-chloro-ethyl)-3-cyclohexyl-1-nitrosourea) | | |
| Meclorethamine, nitrogen mustard | Mustargen | Merck |
| (2-chloro-N-(2-chloroethyl)-N-methylethanamine | | |
| hydrochloride) | | |
| Megestrol acetate | Megace | Bristol-Myers Squibb |
| 17α(acetyloxy)-6-methylpregna-4,6-diene- | | |
| 3,20-dione | | |
| Melphalan, L-PAM | Alkeran | GlaxoSmithKline |
| (4-(bis(2-chloroethyl) amino)-L-phenylalanine) | | |
| Mercaptopurine, 6-MP | Purinethol | GlaxoSmithKline |
| (1,7-dihydro-6H-purine-6-thione monohydrate) | | |
| Mesna | Mesnex | Asta Medica |
| (sodium 2-mercaptoethane sulfonate) | | |
| Methotrexate | Methotrexate | Lederle Laboratories |
| (N-(4-(((2,4-diamino-6- | | |
| pteridinyl)methyl)methylamino)benzoyl)-L- | | |
| glutamic acid) | | |
| Methoxsalen | Uvadex | Therakos, Inc., Way |
| (9-methoxy-7H-furo(3,2-g)(1)-benzopyran-7-one) | | Exton, Pa |
| Mitomycin C | Mutamycin | Bristol-Myers Squibb |
| mitomycin C | Mitozytrex | SuperGen, Inc., |
| | | Dublin, CA |
| Mitotane | Lysodren | Bristol-Myers Squibb |
| (1,1-dichloro-2-(o-chlorophenyl)-2-(p- | | |
| chlorophenyl) ethane) | | |
| Mitoxantrone | Novantrone | Immunex |
| (1,4-dihydroxy-5,8-bis((2-((2- | | Corporation |
| hydroxyethyl)amino)ethyl)amino)-9,10- | | |
| anthracenedione dihydrochloride) | | |
| Nandrolone phenpropionate | Durabolin-50 | Organon, Inc., West |
| | | Orange, NJ |
| Nofetumomab | Verluma | Boehringer Ingelheim |
| | | Pharma KG, |
| | | Germany |
| Oprelvekin | Neumega | Genetics Institute, |
| (IL-11) | | Inc., Alexandria, VA |
| Oxaliplatin | Eloxatin | Sanofi Synthelabo, |
| (cis-((1R,2R)-1,2-cyclohexanediamine-N,N′) | | Inc., NY, NY |
| (oxalato(2-)-O,O′) platinum) | | |
| Paclitaxel | TAXOL | Bristol-Myers Squibb |
| (5β,20-Epoxy-1,2a,4,7β,10β,13a- | | |
| hexahydroxytax-11-en-9-one 4,10-diacetate 2- | | |
| beuzoate 13-ester with (2R,3S)-N-benzoyl-3- | | |
| phenylisoserine) | | |
| Pamidronate | Aredia | Novartis |
| (phosphonic acid (3-amino-1-hydroxypropylidene) | | |
| bis-, disodium salt, pentahydrate, (APD)) | | |
| Pegademase | Adagen | Enzon |
| ((monomethoxypolyethylene glycol succinimidyl) | (Pegademase | Pharmaceuticals, Inc., |
| 11-17-adenosine deaminase) | Bovine) | Bridgewater, NJ |
| Pegaspargase | Oncaspar | Enzon |
| (monomethoxypolyethylene glycol succinimidyl | | |
| L-asparaginase) | | |
| Pegfilgrastim | Neulasta | Amgen, Inc |
| (covalent conjugate of recombinant methionyl | | |
| human G-CSF (Filgrastim) and | | |
| monomethoxypolyethylene glycol) | | |
| Pentostatin | Nipent | Parke-Davis |
| | | Pharmaceutical Co., |
| | | Rockville, MD |
| Pipobroman | Vercyte | Abbott Laboratories, |
| | | Abbott Park, IL |
| Plicamycin, Mithramycin | Mithracin | Pfizer, Inc., NY, NY |
| (antibiotic produced by Streptomyces plicatus) | | |
| Porfimer sodium | Photofrin | QLT |
| | | Phototherapeutics, |
| | | Inc., Vancouver, |
| | | Canada |
| Procarbazine | Matulane | Sigma Tau |
| (N-isopropyl-μ-(2-methylhydrazino)-p-toluamide | | Pharmaceuticals, Inc., |
| monohydrochloride) | | Gaithersburg, MD |
| Quinacrine | Atabrine | Abbott Labs |
| (6-chloro-9-(1-methyl-4-diethyl-amine) | | |
| butylamino-2-methoxyacridine) | | |
| Rasburicase | Elitek | Sanofi-Synthelabo, |
| (recombinant peptide) | | Inc., |
| Rituximab | Rituxan | Genentech, Inc., |
| (recombinant anti-CD20 antibody) | | South San Francisco, |
| | | CA |
| Sargramostim | Prokine | Immunex Corp |
| (recombinant peptide) | | |
| Streptozocin | Zanosar | Pharmacia & Upjohn |
| (streptozocin 2-deoxy-2- | | Company |
| (((methylnitrosoamino)carbonyl)amino)-a(and b)- | | |
| D-glucopyranose and 220 mg citric acid | | |
| anhydrous) | | |
| Talc | Sclerosol | Bryan, Corp., |
| (Mg₃Si₄O₁₀(OH)₂) | | Woburn, MA |
| Tamoxifen | Nolvadex | AstraZeneca |
| ((Z)2-(4-(1,2-diphenyl-1-butenyl) phenoxy)-N,N- | | Pharmaceuticals |
| dimethylethanamine 2-hydroxy-1,2,3- | | |
| propanetricarboxylate (1:1)) | | |
| Temozolomide | Temodar | Schering |
| (3,4-dihydro-3-methyl-4-oxoimidazo(5,1-d)-as- | | |
| tetrazine-8-carboxamide) | | |
| teniposide, VM-26 | Vumon | Bristol-Myers Squibb |
| (4′-demethylepipodophyllotoxin 9-(4,6-0-(R)-2- | | |
| thenylidene-(beta)-D-glucopyranoside)) | | |
| Testolactone | Teslac | Bristol-Myers Squibb |
| (13-hydroxy-3-oxo-13,17-secoandrosta-1,4-dien- | | |
| 17-oic acid (dgr)-lactone) | | |
| Thioguanine, 6-TG | Thioguanine | GlaxoSmithKline |
| (2-amino-1,7-dihydro-6H-purine-6-thione) | | |
| Thiotepa | Thioplex | Immunex |
| (Aziridine, 1,1′,1″-phosphinothioylidynetris-, or | | Corporation |
| Tris (1-aziridinyl) phosphine sulfide) | | |
| Topotecan HCl | Hycamtin | GlaxoSmithKline |
| ((S)-10-((dimethylamino) methyl)-4-ethyl-4,9- | | |
| dihydroxy-1H-pyrano(3′,4′:6,7) indolizino (1,2-b) | | |
| quinoline-3,14-(4H,12H)-dione | | |
| monohydrochloride) | | |
| Toremifene | Fareston | Roberts |
| (2-(p-((Z)-4-chloro-1,2-diphenyl-1-butenyl)- | | Pharmaceutical |
| phenoxy)-N,N-dimethylethylamine citrate (1:1)) | | Corp., Eatontown, NJ |
| Tositumomab, I 131 Tositumomab | Bexxar | Corixa Corp., Seattle, |
| (recombinant murine immunotherapeutic | | WA |
| monoclonal IgG₂ₐ lambda anti-CD20 antibody (I | | |
| 131 is a radioimmunotherapeutic antibody)) | | |
| Trastuzumab | Herceptin | Genentech, Inc |
| (recombinant monoclonal IgG₁ kappa anti-HER2 | | |
| antibody) | | |
| Tretinoin, ATRA | Vesanoid | Roche |
| (all-trans retinoic acid) | | |
| Uracil Mustard | Uracil Mustard | Roberts Labs |
| | Capsules | |
| Valrubicin, N-trifluoroacetyladriamycin-14- | Valstar | Anthra --> Medeva |
| valerate | | |
| ((2S-cis)-2-(1,2,3,4,6,11-hexahydro-2,5,12- | | |
| trihydroxy-7 methoxy-6,11-dioxo-((4 2,3,6- | | |
| trideoxy-3-((trifluoroacetyl)-amino-α-L-lyxo- | | |
| hexopyranosyl)oxyl)-2-naphthacenyl)-2-oxoethyl | | |
| pentanoate) | | |
| Vinblastine, Leurocristine | Velban | Eli Lilly |
| (C₄₆H₅₆N₄O₁₀.H₂SO₄) | | |
| Vincristine | Oncovin | Eli Lilly |
| (C₄₆H₅₆N₄O₁₀.H₂SO₄) | | |
| Vinorelbine | Navelbine | GlaxoSmithKline |
| (3′,4′-didehydro-4′-deoxy-C′- | | |
| norvincaleukoblastine (R-(R*,R*)-2,3- | | |
| dihydroxybutanedioate(1:2)(salt))) | | |
| Zoledronate, Zoledronic acid | Zometa | Novartis |
| ((1-Hydroxy-2-imidazol-1-yl-phosphonoethyl) | | |
| phosphonic acid monohydrate) | | |

Preferred conventional anticancer agents for use in administration with the disclosed dsRNA compositions include, but are not limited to, adriamycin, 5-fluorouracil, etoposide, camptothecin, actinomycin-D, mitomycin C, cisplatin, docetaxel, gemcitabine, carboplatin, oxaliplatin, bortezomib, gefitinib, and bevacizumab. These agents can be prepared and used singularly, in combined therapeutic compositions, in kits, or in combination with immunotherapeutic agents, and the like.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXPERIMENTAL

The following examples are provided in order to demonstrate and further illustrate certain preferred embodiments and aspects of the present invention and are not to be construed as limiting the scope thereof.

In the experimental disclosure which follows, the following abbreviations apply: Invitrogen (Invitrogen Corp., Carlsbad, Calif.); Calbiochem (EMD Biosciences, Inc, San Diego, Calif.); Sigma (Sigma Chemical Co., St. Louis, Mo.);, GenScript (GenScript, Piscataway, N.J.; Qiagen (Qiagen, Hilden, Germany); Roche (F. Hoffmann-La Roche Ltd, Basel, Switzerland); ° C. (degrees Centigrade); cm (centimeters); g (grams); l or L (liters); μg (micrograms); μl (microliters); μm (micrometers); μM (micromolar); μmol (micromoles); mg (milligrams); ml (milliliters); mm (millimeters); mM (millimolar); mmol (millimoles); M (molar); mol (moles); ng (nanograms); nm (nanometers); nmol (nanomoles); N (normal); pmol (picomoles);

### EXAMPLE 1

### Sequences Encoding siRNA for Bcl-xL

Sequences encoding siRNA for Bcl-xL. The sequences of dsRNA are designed based on a 189-nt mRNA sequence from Bcl-xL gene (Accession number: NM_{—}138578) (783-931) that is spliced out and therefore not present in alternately spliced Bcl-xS mRNA (Accession number: Z23116). The dsRNA sequences chosen are designed to target the alternately spliced region in Bcl-xL mRNA, such that they do not hybridize to Bcl-xS mRNA. Bcl-xS is considered an antagonist of Bcl-xL and a promotor of cell death. Sequences encoding dsRNA (and siRNAs derived therefrom) that specifically down-regulate Bcl-xL but not Bcl-xS were determined. 7 siRNA sequences were chosen using GenScript's siRNA Target Finder, followed by GenScript's siRNA Construct Builder to design small hairpin cassettes (www.GenScript.com). The sequences are as follows:

siRNA #1 (SEQ ID NO: 3): 75 bp.
GGATCCCGCGATCCGACTCACCAATACCTTGATATCCGGGTATTGGTGAG
TCGGATCGCTTTTTTCCAAAAGCTT
siRNA #2 (SEQ ID NO: 4):: 76 bp.
GGATCCCGCTTGTCTACGCTTTCCACGCATTGATATCCGTGCGTGGAAAG
CGTAGACAAGTTTTTTCCAAAAGCTT
siRNA #3 (SEQ ID NO: 5):: 76 bp.
GGATCCCGCGATCCGACTCACCAATACCTTTGATATCCGAGGTATTGGTG
AGTCGGATCGTTTTTTCCAAAAGCTT
siRNA #4 (SEQ ID NO: 6):: 75 bp.
GGATCCCAGGTGGTCATTCAGGTAAGTGTTGATATCCGCACTTACCTGAA
TGACCACCTTTTTTTCCAAAAGCTT
siRNA #5 (SEQ ID NO: 7):: 76 bp.
GGATCCCGCAAAGCTCTGATATGCTGTCCTTGATATCCGGGACAGCATAT
CAGAGCTTTGTTTTTTCCAAAAGCTT
siRNA #6 (SEQ ID NO: 8):: 75 bp.
GGATCCCAGCTCTGATATGCTGTCCCTGTTGATATCCGCAGGGACAGCAT
ATCAGAGCTTTTTTTCCAAAAGCTT
siRNA #7 (SEQ ID NO: 9):: 76 bp.
GGATCCCGCCTTGTCTACGCTTTCCACGCTTGATATCCGGCGTGGAAAGC
GTAGACAAGGTTTTTTCCAAAAGCTT

### EXAMPLE 2

### Transfection of Tumor Cells With Bcl-xL siRNA Cassettes

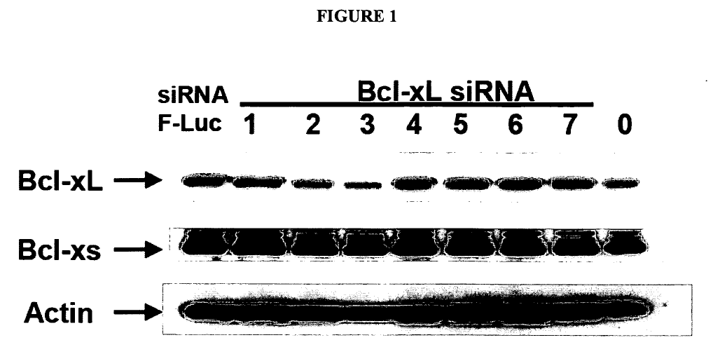

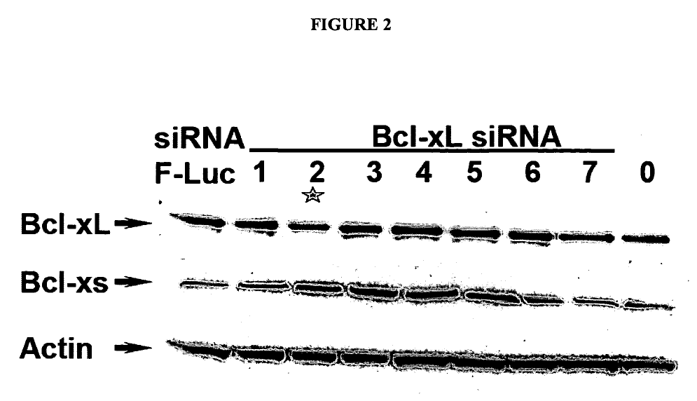

Human prostate cancer PC-3 cells and human breast cancer MCF-7 cells were transfected with the designed Bcl-xL dsRNA (siRNA) cassettes, using a transferrin-liposome system (LipofectaminePlus, Invitrogen). Briefly, 5×10⁵ cells were plated per well in 6-well plates. After overnight culture, the cells were 60% to 70% confluent. The transfection was performed according to the manufacturer's instruction, with 0.4 μg siRNA cassette DNA per well. The cells were cultured for 48 hr, then harvested and cell lysates were made for Western blot analysis. 15 μg of protein were added to each lane for SDS-PAGE. The blot membrane was probed with antibodies against human Bcl-xL (Calbiochem), Bcl-xS (Calbiochem) and β-actin (Sigma), respectively. siRNA #2 and #3 down-regulated Bcl-xL expression in PC-3 cells up to 50%, as compared to the siRNA to firefly luciferase gene (F-Luc) or liposome control (Lane “0”), without significantly affecting Bcl-xS expression (See, e.g., FIG. 1). The transfection efficiency of LipofectaminePlus was 40% to 50%, indicating that the Bcl-xL gene knockdown by siRNA #2 and #3 were close to 100% in the cells transfected. Similar results were also observed in MCF-7 cells (See, e.g., FIG. 2). The transfection efficiency of LipofectaminePlus in MCF-7 cells was also 40% to 50%. siRNA #2 showed the best down-regulation, up to >50% down-regulation of Bcl-xL, without significantly affecting Bcl-xS expression. Considering the transfection efficiency is 40% -50%, this 50% down-regulation indicates that, in the transfected cells, Bcl-xL down-regulation is close to 100%. FIG. 3 shows the photos of MCF-7 cells 48 hr after transfection. Transfection of BclxL siRNA #2 resulted in up to 50% cell death, Bcl-xL siRNA #3 induced somewhat less cell death, whereas control siRNA and others caused no significant cell death, corresponding to the levels of down-regulation of Bcl-xL. Therefore, with efficient delivery to the cells, the Bcl-xL siRNA effectively induces cell death.

### EXAMPLE 3

### Construction of psiBcl-xL for Therapeutic Applications.

Based on in vitro transfection data, siRNA #2, that displayed near complete down-regulation of Bcl-xL while at the same time not affecting Bcl-xS levels, was chosen for constructing a plasmid based siRNA vector. pRNATU6.1/Hygro was purchased from GenScript Corporation. siRNA #2 was inserted into the pRNATU6.1Hygro plasmid via BamH I and Hind III sites with the cloned product designated psiBcl-xL (See, e.g., FIG. 4). The construct was confirmed by sequencing and the plasmid was propagated in E. coli and purified with QiaGene Maxi kit. The same vector carrying siRNA for firefly luciferase gene (F-Luc), (designated psiLuc), was obtained from GenScript and used as vector control.

### EXAMPLE 4

### Transfection of Tumor Cells With Bcl-xL siRNA Vector, psiBcl-xL

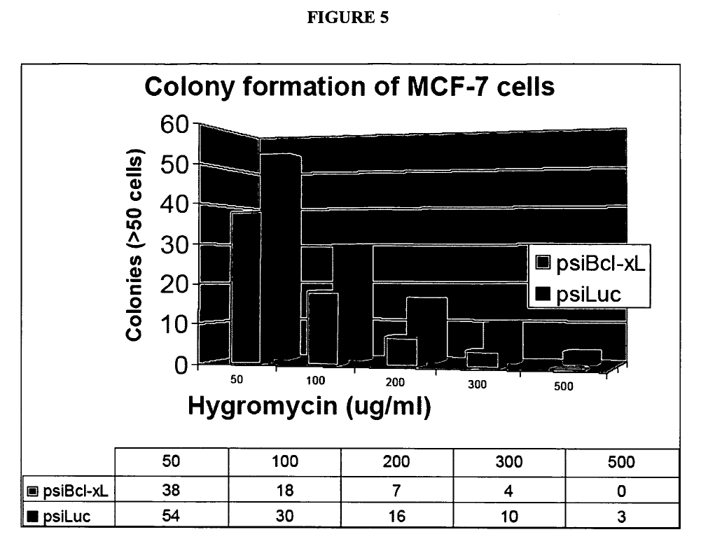

MCF-7 and PC-3 cells were transfected with psiBcl-xL (transfection conditions were performed as described above). In some experiments, FuGene 6 (Roche) was used due to low toxicity and high efficiency for plasmid transfection. Briefly, 60% confluent cells were transfected with 1-3 μg psiBcl-xL or psiLuc DNA per well in a 6-well plate (DNA:FuGene 6 ratio=1 μg:3 μl). After 48 hr of cell culture, the cells were collected, and lysed with lysates prepared for Western blot analysis, or trypsinized and replated into 6-well plates, and 50-800 ng/ml hygromycin (Invitrogen) was added for stable clone selection. The transfection efficiency of FuGene 6 (as detected by green fluorescence protein (GFP) expression; psiBcl-xL posseses the green fluorescence protein (GFP) gene, See, e.g., FIG. 4) for MCF-7 cells was about 40% -50%. MCF-7 cells transfected with psiBcl-xL showed significant cell death (up to 50%), similar to that observed in cells transfected with Bcl-xL siRNA #2 cassette shown above (See, e.g., FIG. 3). For stable clone selection, MCF-7 cells were transfected with psiLuc and psiBcl-xL, then the cells were cultured in selection media with various concentrations of hygromycin for three weeks. The colony formation of MCF-7 stable clones is shown (See, e.g., FIG. 5).

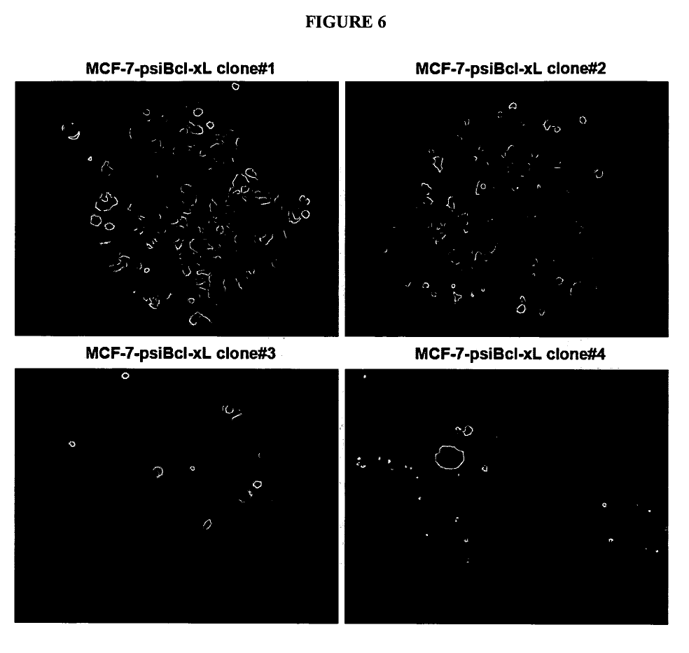

At different doses of hygromycin, there are more psiLuc clones than psiBcl-xL clones, indicating that Bcl-xL down-regulation renders the cells more sensitive to the cytotoxicity of hygromycin. The untransfected MCF-7 control cells show no colony formation at hygromycin doses>200 μg/ml. 300 μg/ml hygromycin dose was chosen as the optimal selection dose, and 4 clones with strong green fluorescence were picked (See, e.g., FIG. 6, depicting the fluorescent photos of MCF-7-psiBcl-xL stable clones). The different MCF-7-psiBcl-xL clones have different levels of green fluorescence protein (GFP) expression. The level of GFP expression should be proportional to the copy number of psiBcl-xL stably transfected in the cells, which in turn corresponds to the level of Bcl-xL siRNA transcription from the plasmid psiBcl-xL.

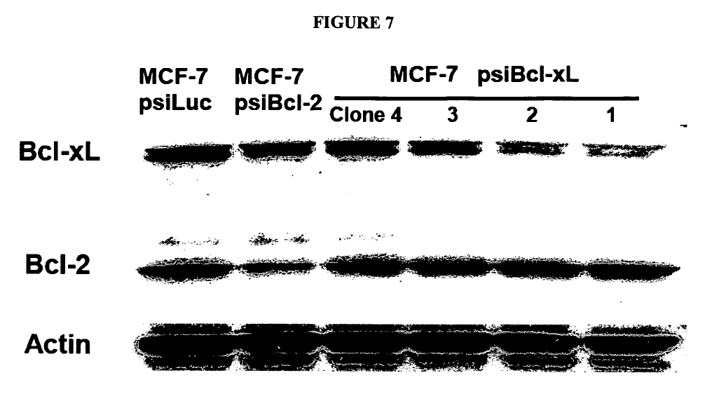

Analysis of Western blot data of MCF-7 psiBcl-xL stable clones shows that the MCF-7-psiBcl-xL clones #1 and #2 display >95% and >90% down-regulation of Bcl-xL gene expression, respectively, whereas no significant change is observed for Bcl-xS and Bcl-2 expression (See, e.g., FIG. 7). Thus, clones #1 and #2 have the strongest downregulation of Bcl-xL, while these two clones also have the strongest green fluorescence. Clone #3 showed >60% down-regulation of Bcl-xL and has moderate green fluorescence, while clone #4 showed no significant Bcl-xL-downregulation together with poor green fluorescence. It is note that weaker inhibitors (e.g., clone #3) may find particular use in research and drug screening applications.

### EXAMPLE 5

### siRNA-Mediated Down-Regulation of Bcl-xL Results in Sensitization of Tumor Cells to Chemotherapy

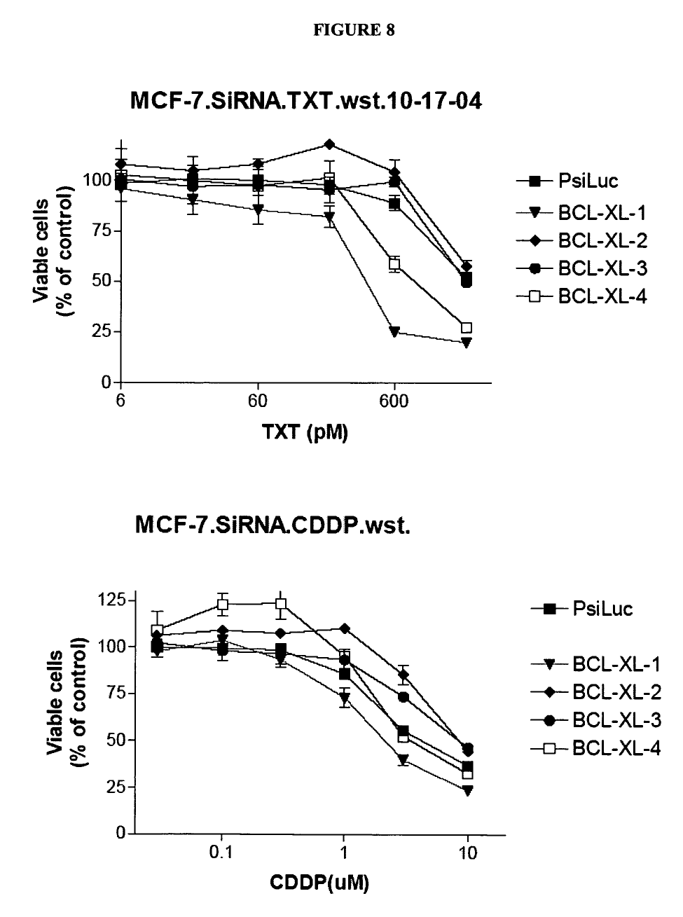

Overexpression of anti-apoptotic protein Bcl-xL renders cancer cells more resistance to chemotherapeutic agents. Down-regulation of Bcl-xL by psiBcl-xL overcomes this apoptosis-resistance, thus enhancing the induction of cell death by chemotherapy. MCF-7 psiBcl-xL Clone #1 showed 5-fold more sensitivity to docetaxel (TXT)-induced cell death than did the control psiLuc clone (See, e.g., FIG. 8). Clone #1 also showed a moderate sensitization to cisplatin (CDDP), (See, e.g., FIG. 8B). As seen above, Clone #1 has the best Bcl-xL gene knockdown (>95%, See, e.g., FIG. 7).

### EXAMPLE 6

### psiBcl-xL-Transfection Results in Loss of Tumorigenecity of MCF-7 Cells

5×10⁷ MCF-7 psiBcl-xL stable clone cells were inoculated in the mammary fat pad of ovariectomized female nude mice (Ncr-nu/nu), 24 hr after the E2 pellets were put in. The tumor take rate was recorded and tumor sizes measured. Table 1 summarizes the tumorigenecity results 19 days after inoculation. MCF-7-psiLuc clone cells retain good tumorigenecity, with tumor take rate ⅚ (5 tumors out of 6 inoculations), comparable with the tumor take rate of untransfected, original MCF-7 cells. Clone #1 (MCF-7-psiBcl-xL-1) did not show any sign of xenograft tumor growth 19 days after inoculation ( 0/6 or 0 tumors out of 6 inoculations). Clones #3 and #4 have only one tumor, tumor take rate ⅙. Clone #2 generated 4 tumors out of 6 inoculations (take rate ⅙). This is consistent with chemoresistance data in FIG. 8, that Clone #2 is not sensitive to TXT and CDDP, although Bcl-xL is down-regulated in this clone (See, e.g., FIG. 7). A possible explanation is that this clone may have other anti-apoptotic protein(s) up-regulated during the selection process to compensate the down-regulated Bcl-xL. Such proteins may include Mcl-1, IAP family proteins, AKT, STAT3/5, etc.

TABLE 1
Tumorigenecity of MCF-7-psiBcl-xL stable clones in nude mice
(Day 19)

| | | psiBcl- | psiBcl- | psiBcl- | psiBcl- |
|---|---|---|---|---|---|
| Mouse # | psiLuc | xL-1 | xL-2 | xL-3 | xL-4 |
| 1 | 21.44 | 0 | 46.4 | 0 | 0 |
| 2 | 23.28 | 0 | 0 | 0 | 0 |
| 3 | 226.48 | 0 | 48.749 | 0 | 0 |
| 4 | 72.48 | 0 | 127.87 | 13.50 | 0 |
| 5 | 51.77 | 0 | 0 | 0 | 46.23 |
| 6 | 0 | 0 | 7.7175 | 0 | 0 |
| Mean | 65.91 | 0.00 | 38.46 | 2.25 | 7.70 |
| SD | 82.6625 | 0 | 49.125349 | 5.5113519 | 18.872298 |

Values are tumor sizes (mm3).

All publications and patents mentioned in the above specification are herein incorporated by reference. Various modifications and variations of the described compositions and methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

We claim:

1. A composition comprising dsRNA, wherein said dsRNA comprises a sense RNA strand and an antisense RNA strand, wherein said sense and said antisense RNA strands form an RNA duplex, and wherein said duplex comprises a complementary nucleotide sequence that is selectively complementary to at least a part of bcl-xL mRNA sequence or equivalent sequences but not selectively complementary to bcl-xS mRNA sequence or equivalent sequences.

2. The composition of claim 1, wherein said complementary nucleotide sequence is substantially identical to a target sequence of about 19 to about 25 contiguous nucleotides in bcl-xL mRNA.

3. The composition of claim 1, wherein said sense RNA strand comprises one RNA molecule and said antisense RNA strand comprises one RNA molecule.

4. The composition of claim 1, wherein said RNA duplex comprises at least one linker between said sense and said antisense RNA strands.

5. The composition of claim 4, wherein said sense and said antisense RNA strands forming said RNA duplex are linked via a single-stranded hairpin.

6. The composition of claim 4, wherein said linker is a chemical linker.

7. The composition of claim 1, wherein said dsRNA comprises non-nucleotide material.

8. The composition of claim 1, wherein said sense and said antisense RNA strands are stabilized against nuclease degradation.

9. The composition of claim 1, wherein at least one of said RNA strands comprises a nucleotide overhang.

10. The composition of claim 9, wherein said overhang is a 3′ overhang.

11. A recombinant nucleic acid comprising a vector comprising nucleic acid sequence for expressing a dsRNA comprising a sense RNA strand and an antisense RNA strand, wherein said sense and said antisense RNA strands form an RNA duplex, and wherein said duplex comprises a complementary nucleotide sequence that is selectively complementary to at least a part of bcl-xL mRNA sequence or equivalent sequences but not selectively complementary to bcl-xS mRNA sequence or equivalent sequences.

12. The recombinant nucleic acid of claim 11, wherein said vector is selected from the group consisting of a plasmid, an adenoviral vector, an adeno-associated vector, a lentiviral vector, a retroviral vector, and a herpes virus vector.

13. The recombinant nucleic acid of claim 11, wherein said nucleic acid sequence for expressing a dsRNA comprises an inducible or regulatable promoter.

14. The recombinant nucleic acid of claim 11, wherein said vector comprises a human H1 or human U6 promoter.

15. The recombinant nucleic acid of claim 11, wherein said nucleic acid sequence for expressing a dsRNA comprises a sense RNA strand coding sequence in operable combination with i) a human U6 promoter and ii) a poly T termination sequence; and an antisense RNA strand coding sequence in operable combination with i) a human U6 promoter and ii) a poly T termination sequence.

16. The recombinant nucleic acid of claim 11, wherein said recombinant nucleic acid is present within a pharmaceutical composition comprising a pharmaceutically acceptable carrier.

17. A method of modifying levels of human bcl-xL mRNA, while concurrently unaffecting levels of bcl-xS mRNA, comprising administering to a host or host tissue an effective amount of a dsRNA comprising a sense RNA strand and an antisense RNA strand, wherein said sense and said antisense RNA strands form an RNA duplex, and wherein said duplex comprises a complementary nucleotide sequence that is selectively complementary to at least a part of bcl-xL mRNA sequence or equivalent sequences but not selectively complementary to bcl-xS mRNA sequence or equivalent sequences.

18. The method of claim 17, wherein said modifying reduces levels of bcl-xL mRNA.

19. The method of claim 17, wherein said host is a human being.

20. The method of claim 17, wherein said effective amount of said dsRNA is from about 1 nM to about 100 nM.


SEQUENCE LISTING (10 sequences)

SEQ ID NO: 1 - DNA, 2575 bp, Homo sapiens
ggaggaggaagcaagcgagggggctggttcctgagcttcgcaattcctgtgtcgccttctgggctcccagcctgccgggtcgcatgatccctccggccggagctggtttttttgccagccaccgcgaggccggctgagttaccggcatccccgcagccacctcctctcccgacctgtgatacaaaagatcttccgggggctgcacctgcctgcctttgcctaaggcggatttgaatctctttctctcccttcagaatcttatcttggctttggatcttagaagagaatcactaaccagagacgagactcagtgagtgagcaggtgttttggacaatggactggttgagcccatccctattataaaaatgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactggccacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggtagtgaatgaactcttccgggatggggtaaactggggtcgcattgtggcctttttctccttcggcggggcactgtgcgtggaaagcgtagacaaggagatgcaggtattggtgagtcggatcgcagcttggatggccacttacctgaatgaccacctagagccttggatccaggagaacggcggctgggatacttttgtggaactctatgggaacaatgcagcagccgagagccgaaagggccaggaacgcttcaaccgctggttcctgacgggcatgactgtggccggcgtggttctgctgggctcactcttcagtcggaaatgaccagacactgaccatccactctaccctcccacccccttctctgctccaccacatcctccgtccagccgccattgccaccaggagaaccactacatgcagcccatgcccacctgcccatcacagggttgggcccagatctggtcccttgcagctagttttctagaatttatcacacttctgtgagacccccacacctcagttcccttggcctcagaattcacaaaatttccacaaaatctgtccaaaggaggctggcaggtatggaagggtttgtggctgggggcaggagggccctacctgattggtgcaacccttaccccttagcctccctgaaaatgtttttctgccagggagcttgaaagttttcagaacctcttccccagaaaggagactagattgcctttgttttgatgtttgtggcctcagaattgatcattttccccccactctccccacactaacctgggttccctttccttccatccctaccccctaagagccatttaggggccacttttgactagggattcaggctgcttgggataaagatgcaaggaccaggactccctcctcacctctggactggctagagtcctcactcccagtccaaatgtcctccagaagcctctggctagaggccagccccacccaggagggagggggctatagctacaggaagcaccccatgccaaagctagggtggcccttgcagttcagcaccaccctagtcccttcccctccctggctcccatgaccatactgagggaccaactgggcccaagacagatgccccagagctgtttatggcctcagctgcctcacttcctacaagagcagcctgtggcatctttgccttgggctgctcctcatggtgggttcaggggactcagccctgaggtgaaagggagctatcaggaacagctatgggagccccagggtcttccctacctcaggcaggaagggcaggaaggagagcctgctgcatggggtggggtagggctgactagaagggccagtcctgcctggccaggcagatctgtgccccatgcctgtccagcctgggcagccaggctgccaaggccagagtggcctggccaggagctcttcaggcctccctctctcttctgctccacccttggcctgtctcatccccaggggtcccagccaccccgggctctctgctgtacatatttgagactagtttttattccttgtgaagatgatatactatttttgttaagcgtgtctgtatttatgtgtgaggagctgctggcttgcagtgcgcgtgcacgtggagagctggtgcccggagattggacggcctgatgctccctcccctgccctggtccagggaagctggccgagggtcctggctcctgaggggcatctgcccctcccccaacccccaccccacacttgttccagctctttgaaatagtctgtgtgaaggtgaaagtgcagttcagtaataaactgtgtttactcagtgaaaaaaaaaaaaaaaaaa

SEQ ID NO: 2 - DNA, 737 bp, Homo sapiens
gaatctctttctctcccttcagaatcttatcttggctttggatcttagaagagaatcactaaccagagacgagactcagtgagtgagcaggtgttttggacaatggactggttgagcccatccctattataaaaatgtctcagagcaaccgggagctggtggttgactttctctcctacaagctttcccagaaaggatacagctggagtcagtttagtgatgtggaagagaacaggactgaggccccagaagggactgaatcggagatggagacccccagtgccatcaatggcaacccatcctggcacctggcagacagccccgcggtgaatggagccactggccacagcagcagtttggatgcccgggaggtgatccccatggcagcagtaaagcaagcgctgagggaggcaggcgacgagtttgaactgcggtaccggcgggcattcagtgacctgacatcccagctccacatcaccccagggacagcatatcagagctttgaacaggatacttttgtggaactctatgggaacaatgcagcagccgagagccgaaagggccaggaacgcttcaaccgctggttcctgacgggcatgactgtggccggcgtggttctgctgggctcactcttcagtcggaaatgaccagacactgaccatccactctaccctcccacccccttctctgctccaccacatcctccgtccagccgccattgccaccaggagaacccg

SEQ ID NO: 3 - DNA, 75 bp, Homo sapiens
ggatcccgcgatccgactcaccaataccttgatatccgggtattggtgagtcggatcgcttttttccaaaagctt

SEQ ID NO: 4 - DNA, 76 bp, Homo sapiens
ggatcccgcttgtctacgctttccacgcattgatatccgtgcgtggaaagcgtagacaagttttttccaaaagctt

SEQ ID NO: 5 - DNA, 76 bp, Homo sapiens
ggatcccgcgatccgactcaccaatacctttgatatccgaggtattggtgagtcggatcgttttttccaaaagctt

SEQ ID NO: 6 - DNA, 75 bp, Homo sapiens
ggatcccaggtggtcattcaggtaagtgttgatatccgcacttacctgaatgaccacctttttttccaaaagctt

SEQ ID NO: 7 - DNA, 76 bp, Homo sapiens
ggatcccgcaaagctctgatatgctgtccttgatatccgggacagcatatcagagctttgttttttccaaaagctt

SEQ ID NO: 8 - DNA, 75 bp, Homo sapiens
ggatcccagctctgatatgctgtccctgttgatatccgcagggacagcatatcagagctttttttccaaaagctt

SEQ ID NO: 9 - DNA, 76 bp, Homo sapiens
ggatcccgccttgtctacgctttccacgcttgatatccggcgtggaaagcgtagacaaggttttttccaaaagctt

SEQ ID NO: 10 - DNA, 64 bp, Homo sapiens
ggatccgagctcggtaccaagcttaagtttaaaccgctgatcagcctcgactgtgccttctagt